(19) United States
(12) Patent Application Publication
(10) Pub. No.: US 2010/0105061 A1
(43) Pub. Date: Apr. 29, 2010

(54) AUTOIMMUNE GENES IDENTIFIED IN SYSTEMIC LUPUS ERYTHEMATOSUS (SLE)

(75) Inventors: Chaim O. JACOB, Los Angeles, CA (US); Raphael ZIDOVETZKI, Los Angeles, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/608,865

(22) Filed: Oct. 29, 2009

Related U.S. Application Data
(60) Provisional application No. 61/109,488, filed on Oct. 29, 2008.
(60) Provisional application No. 61/164,897, filed on Mar. 30, 2009.

(51) Int. Cl. C12Q 1/68

(52) U.S. Cl. 435/6

(57) ABSTRACT

The present invention relates to a genes and autoimmunity. More specifically, the invention relates to the identification of genes that are associated with Systemic Lupus Erythematosus (SLE) in children and/or adults. Also disclosed are methods for making and using the genetic selection tool used to identify SLE associated genes, and their uses in diagnosis and treatment of the disease.

The present application claims the benefit of the filing date of U.S. Provisional Application No. 61/109,488, filed Oct. 29, 2008, and U.S. Provisional Application No. 61/164,897, filed Mar. 30, 2009, the disclosure of which is incorporated herein by reference in its entirety.

### STATEMENT REGARDING FEDERALLY SPONSORED RESEARCH OR DEVELOPMENT

This invention was made with government support under Contract No. RO1 AR 445650 awarded by the National Institutes of Health. The government has certain rights in the invention.

### FIELD OF THE INVENTION

The present invention relates in general to autoimmune diseases. More specifically, the invention provides a device for identifying genes and their variants associated with Systemic Lupus Erythematosus.

### BACKGROUND OF THE INVENTION

Systemic lupus erythematosus (SLE) is a debilitating multisystem autoimmune disorder affecting approximately 0.1% of the North American population (predominantly females), characterized by chronic inflammation in multiple organ systems and the production of autoantibodies to a multitude of self-antigens (1). The prevalence of SLE varies among ethnic population (higher in non-Caucasians) and is likely attributable to ethnic/race differences in genetic susceptibility and complex interactions with environmental exposures. Genome-wide linkage studies performed in relatively small collections of families with two or more affected members have identified several genetic intervals (2), suggesting that multiple genes contribute to the pathogenesis of SLE. While the linkage analyses employed are quite successful in identifying rare variants with strong genetic effects, this approach has limited power to detect common-variants with more modest effects. Although some rare alleles with strong genetic effects (such as C1q deficiency) can contribute to SLE genetics, it is probable that common alleles with modest genetic effects play a more important role in disease susceptibility. Thus, many genetic alleles important for the SLE phenotype have been missed through genome-wide linkage studies. As a case in point, an allele of PTPN22, a lymphocytic phosphatase, is a risk factor for SLE (3). PTPN22 is encoded on chromosome 1 at 1p12, a region which was not picked up by any of the SLE linkage studies.

Since association studies are more powerful than linkage studies when the predisposing variant is more frequent (4), a better strategy is to perform a series of candidate gene single nucleotide polymorphisms (SNP) screens in a study population that is most conducive to express these susceptibility genes. However, it is becoming increasingly clear that association studies performed on a wide random selection of genes are unlikely to provide reproducible results; indeed it has been estimated that the rate of false positive results reported by such studies is near 95% (5). Accordingly, it has been suggested that a Bayesian methodology (wherein instead of selecting candidate genes at random, the investigators select the candidate genes based on prior available information), will increase the statistical power of finding genes actually associated with disease (5, 6). Based on these considerations, we have developed and tested a new gene selection tool which is now standardized and available for the scientific community at large.

Although genetic variations have been already recognized as risk factors for SLE, in most cases it remains unclear whether these polymorphisms represent the causal alleles or rather are simply in linkage disequilibrium with other alleles that might be the ‘real’ causal variants. Without systematic search for all possible polymorphisms within a susceptibility gene it is premature to suggest that a given variant is causal even when functional consequences can be attributable to the variant. Very few systematic searches for all polymorphisms in any lupus gene have been performed to date. With the ongoing development of next generation sequencing technologies it is now possible to evaluate the full range of polymorphisms within a gene in a timely and cost-effective manner as we suggest here. Moreover, the technologies being developed (at an incredibly rapid rate) are such that it becomes much more reasonable to investigate several genes simultaneously than one at a time. We argue that the subsequent step should be a re-examination of all relevant polymorphisms which should have the highest probability to identify the causal variants (or causal haplotype blocks).

At the juncture that all (or at least most) polymorphisms have been identified within a haplotype block and reevaluated in relevant patient populations, systematic functional studies should be most appropriate toward discovering the mechanisms through which the genetic variants are involved in the causation or perpetuation of SLE.

We believe that the step by step approach and the parallel testing of several genes as suggested in the present application, is more efficient and economical than focusing on each gene separately. Furthermore, our success in developing collaborative efforts to assemble a very large population of SLE patients of multiple ethnicities to be tested in parallel will identify those genetic risk factors that are common to multiple ethnic groups. This approach will provide invaluable information regarding differences in disease prevalence and disease progression in different populations and might be able to provide ethnicity-based disease markers and/or diagnostics. Finally, the discovery and characterization of the role of new SLE-associated genes will provide the immediate justification for the development of therapeutic approaches targeting these relevant molecules or other molecules within their biochemical pathway.

### SUMMARY OF THE INVENTION

In one embodiment, the invention relates to methods of diagnosing an autoimmune disease in a child. The method comprises detecting the presence of 2 or more genes that are associated with child-onset autoimmune disease. More preferably, the method diagnoses child-onset SLE. More preferably, the method detects 2 or more of the genes: SELP, IRAK1, KLRG1, TNFSF4, TNFRSF6, TLR8, or Fas.

In a closely related embodiment, the invention relates to methods of diagnosing an autoimmune disease in an adult. The method comprises detecting the presence of 2 or more genes that are associated with adult-onset autoimmune disease. More preferably, the method diagnoses adult-onset SLE. More preferably, the method detects 2 or more of the genes: IRAK1, KLRG1, TNFSF4, or FAIM.

In yet another embodiment, the invention relates to methods of diagnosing an autoimmune disease in a subject. The method comprises detecting the presence of 2 or more genes that are associated with both child- and adult-onset autoimmune disease. More preferably, the method diagnoses SLE. More preferably, the method detects 2 or more of the genes: SELP, IRAK1, KLRG1, IRF5, STAT4, TNFRSF6, TNFSF4, PTPN22, TLR8, FAIM, IRF5, or NCF2.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. Venn block diagrams of overlap between significant SNPs of adult- and childhood-onset SLE. The numbers shown correspond to the numbers of significant SNPs in adult, childhood, or both cohorts of SLE as indicated.

### DETAILED DESCRIPTION OF THE INVENTION

Studies by our group and others have established that the genetics of systemic lupus erythematosus (SLE) is not dominated by a few major genes; rather, numerous genes contribute to the pathogenesis of SLE, each gene providing modest effects. Based on Bayesian methodology, we developed a gene selection tool to increase the power of genetic association studies, and we devised a novel microarray platform to discover susceptibility genes contributing to SLE. We tested our model in a cohort of childhood-onset SLE families.

The top 20 genes identified in our family trio study of ˜1,200 genes and ˜10,000 SNPs have been replicated in a second, much larger childhood-onset SLE cohort and a very large cohort of adult onset SLE using a set of SNPs for each chosen gene. Together, these studies suggest that the following genes are strong candidates for SLE susceptibility in childhood- and/or adult-onset SLE: SELP, IRAK1, KLRG1, IRF5, STAT4, TNFRSF6, TNFSF4, PTPN22, TLR8 and FAIM. Since IRF5, PTPN22, and STAT4, have been associated with SLE by other groups of investigators, our data present further replication in separate cohorts as well as corroborate the validity of our approach.

The present application concentrates on the candidates in which (to the best of our knowledge) we have made significant original observations, and we are presenting here our evidence to support their candidacy and justify follow up studies, with the ultimate goal of characterizing the causal mutations and the mechanisms of disease involvement of these new genes. For the purpose of these studies we have assembled an unparalleled study population of 6,500 adult-onset and 1,100 childhood-onset cases of SLE and a large multidisciplinary collaborative team of investigators.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Example 1

We have adopted a Bayesian approach, in which rather than initially concentrating on specific candidate genes, we developed a collection of candidate pathways. To this end, we have taken advantage of the accumulated data from genome-wide scans of adult SLE families, candidate gene investigations, information gained from genetics of mouse models of lupus and the gene expression profiling data of human SLE. Based upon our extensive literature search and examination, we selected a list of candidate functional pathways judged to be relevant to the pathogenesis of SLE. Three databases (NCBI, GeneCards, Harvester) were searched using a set of keywords representing these functional pathways. This initial inclusive search resulted in the selection of 6,384 genes. Subsequent analyses excluded genes based on their expression pattern or function in unrelated processes (e.g. believed to only be involved in embryogenesis or expressed in tissues deemed irrelevant), or genes included solely on the basis of their homology to a relevant gene, and genes without any known or predicted function. Genes in the latter two groups were only included if they resided within established linkage peaks. Finally, the number of times a gene was picked up by distinct keywords was scored and utilized in prioritization (pathway score). Using these criteria a final list of 1,204 genes was selected.

The choice of SNPs within the selected genes was based on available information from databases and accumulating information from the Human Haplotype Mapping Project (HapMap). Priority was given to SNPs demonstrating high heterozygosity, those that were informative in two or more relevant ethnicities and to SNPs representing amino acid coding variants. The list of SNPs was then cross-checked against the accumulated SNP validation test results available at ParAllele Biosciences from the International HapMap project. A final list of 9,412 SNPs was selected for the genotyping assay development. To this end, the ParAllele molecular inversion probe (MIP) technology on the Affymetrix TAG3 platform was used (7). The genotyping platform was validated on 18 control samples and 5 complete HapMap trios. Of the 9,412 SNPs, robust genotyping data was generated for 9,375 (99.6%). Several control samples were genotyped up to 8 times resulting in a 99.98% repeatability of these genotypes.

The candidate pathway genotyping platform developed was applied to a sample of 753 subjects corresponding to 251 childhood onset SLE trios (patients and both of their parents). Childhood-onset SLE presents a unique subgroup of patients for a genetic study because an earlier disease onset, a more severe disease course, and a greater frequency of family history of SLE (8, 9) may imply an increased likelihood of expressing the genetic etiology. Most previous genetic studies were done in adult onset disease.

The Transmission Disequilibrium Test (TDT) (10) was used to calculate the significance of SNP-association with SLE. The confounding effect due to population stratification is avoided by using the family-based TDT, in which the preferential transmission of the test allele from parents to affected offspring provides evidence for association of the test allele with disease.

We adapted a variation of the false discovery rate (FDR) (11) for the multitest correction. We decided on two levels of FDR as significant outcomes of this study: SNPs with q values (that correspond to probability of the SNP to be a false positive) of less than 0.05 would be considered as “proven” with greater than 95% probability, and q values of less than 0.5 as “noteworthy” which require follow up studies for verification. Table 1 shows that two genes fall into the first category: SELP and IRAK1. Seven additional genes fall into the second category. Thus, the Bayesian design of the microarray and rigorous multitest correction analysis assured that with relatively modest numbers of samples, the design of the study and the analysis resulted in high-confidence findings.

There are a variety of study and analysis designs currently employed in the linkage and association studies, only a few of which utilize rigorous statistical methods (5, 6). It is also quite common that replication studies or even re-analysis of the published data do not confirm the original conclusions. We therefore thought it is important to re-analyze our results using a different statistical approach. Because of the Bayesian methodology used at the outset (during the gene selection for the chip design), a recently published Bayesian data analysis was employed for comparison with the FDR q values analysis: False Positive Report Probability (FPRP) (12). It is remarkable that every gene that was selected using FDR q values analysis method (Table 1) was also found significant in FPRP analysis (7)-demonstrating the robustness of our findings.

Colhoun et al. suggested that in the presence of prior information of association, the p values of 5×10⁻⁴ or smaller can be considered significant (5). Just applying this simple criterion to our TDT results, yields exactly the same genes listed in Table 1.

The top 20 genes in our TDT based family study have been replicated in a second much larger childhood-onset SLE cohort and a very large cohort of adult onset SLE using a case control strategy. Together these studies, suggest that the following genes are strong candidates for susceptibility to SLE in childhood- and/or adult-onset SLE: SELP, IRAK1, KLRG1, IRF5, STAT4, TNFRSF6, TNFSF4, PTPN22, TLR8 and FAIM. Since IRF5, PTPN22 and STAT4, have been associated with SLE by other groups of investigators, our data present further replication in separate cohorts as well as corroborate the validity of our approach. The present application concentrates on the candidates in which we have made significant original observations, and we are presenting here our supporting evidence for their candidacy and to justify follow up studies.

Our replication studies included 11,368 participants (6,066 independent SLE cases and 5,302 healthy controls) enrolled into the Lupus Genetic Study Group at USC and by our collaborators in the Oklahoma Medical Research Foundation (OMRF). Of those 6,066 SLE cases, 769 were defined as childhood-onset according to the criterion that the diagnosis of SLE was made before the age of 13 by at least one pediatric rheumatologist participating in the study. All protocols were approved by the Institutional Review Boards at each respective institution. All patients met the revised 1997 ACR criteria for the classification of SLE. All procedures, methodologies and collection of data were consistent in a standard manner in all participating sites. Self-reported ethnicity was verified by parental and grandparental ethnicity, when possible. Blood samples were collected from each participant, and genomic DNA was isolated and stored using standard methods. Genotyping was performed using Illumina iSelect™ Infinium II Assays on the BeadStation™ 500GX system and the Illumina GoldenGate platform. Genotype data were only used from samples with a call rate greater than 90% of the SNPs screened (98.05% of the samples). The average call rate for all samples was 97.18%. For analysis, only genotype data from SNPs with a call frequency greater than 90% in the samples tested and an Illumina GenTrain score greater than 0.7 were used. In order to minimize sample misidentification, data from 91 SNPs that had been previously genotyped on 42.12% of the samples were used to verify sample identity. In addition, at least one sample previously genotyped was randomly placed on each Illumina Infinium BeadChip and used to track samples throughout the genotyping process.

Testing for association was accomplished using the PLINK and SNPGWA programs (http://www.phs.wfubmc.edu/web/public_bios/sec_gene/downloads.cfm) (13) For each SNP, missing data proportions for cases and controls, minor allele frequency and exact tests for departures from Hardy-Weinberg expectation were calculated.

We have addressed the population stratification confounding by using a set of 233 unlinked ancestry informative markers (AIMS) which were genotyped on the entire sample cohort to estimate the underlying population structure. Using these empirical estimates of population structure we performed final statistical analyses of the data adjusted by the estimated structures. An alternative approach exploiting a principal component analysis according to Price et. al. (55) was used to investigate our results.

The additive model was used as the primary hypothesis of statistical inference, unless the lack of fit (LOF) test for the additive model was significant (LOF p<0.05). If so, then the minimum p-value from the dominant, additive and recessive models was used; for recessive models, at least 30 individuals homozygous for the minor allele were required. Combined p values were calculated from the per-ethnicity p values using the Fisher method. For multiple test correction we calculated the false discovery rate (FDR) q values considering the total number of SNPs tested per gene and the 4 different ethnicities. Q values were calculated using the q value package (available from http://cran.r-project.org) which implements the q value correction of FDR. Table 2 shows that each gene selected for this study contained a number of SNPs with a high degree of significance, after correction for multiple testing.

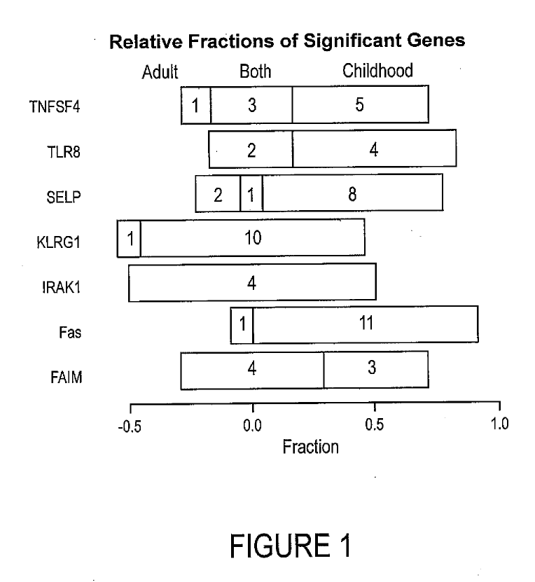

The number of significant SNPs for each gene and overlaps between adult- and childhood-onset analyses are summarized on FIG. 1. TNFSF4 shows significant association with SLE with 9 SNPs. 3 SNPs show association in both adults and children, one SNP is significant only in adults and 5 SNPs are associated only in childhood onset. Table 2 shows, e.g. SNP rs1234315 within the TNFSF4 gene that is associated significantly in adult onset SLE in EA, MA and Asian SLE subjects with p values of 1.71E-5, 7.81E-5 and 4.54E-8 respectively. Similarly significant association has been demonstrated in childhood-onset SLE cases of EA, MA and Asian ethnicities.

Eleven SNPs from SELP out of 31 tested are significantly associated with adult or childhood onset SLE in at least one ethnic group. Eight SNPs show significant association with childhood-onset only; two SNPs that are different from the above, show association with adults-onset SLE in at least one ethnic subpopulation. Thus, although both adult- and childhood-onset SLE subjects show association with SELP, they have distinct sets of SNPs that are associated with the disease. In contrast, 4 SNPs out of 13 tested within the IRAK1 gene show significant association with disease in both childhood- and adult-onset SLE in at least one ethnicity. For example (Table 2), rs763737 is associated with childhood onset SLE in EA, MA and Asians (p=2.5E-3, 3.31E-4 and 5.93E-3 respectively) and also with adult onset SLE especially in Asians (p=6.76E-8). KLRG1 displays significant association with SLE in 11 SNPs. Of these, 10 SNPs show significant association in both adults and children, while a single SNP shows association only in adult-onset SLE at least in one ethnicity.

As depicted in FIG. 1, seven SNPs within (or close to) the FAIM gene were significantly associated in children and/or adults with SLE. The strongest association was demonstrated with SNP rsl13095734 which show association in all 4 ethnicities both in children and adults (for example p=8.7E-14 in MA adults and p=5.09E-26 in MA pediatric SLE). Of the 7 significantly associated SNPs, 4 SNPs were significant in both childhood and adult SLE, while the other 3 SNPs demonstrate significant association only in childhood SLE.

The gene TLR8 shows significant association with 6 SNPs out of 12 tested. Only 2 SNPs have significant association in both adult and childhood SLE (for example SNP rs17256081 p=7.35E-6 in MA adults and 7.63E-5 in MA children). The other 4 SNPs displayed significant association with childhood-onset SLE only.

Our studies demonstrate that TNFRSF6, the apoptosis gene that represents the lpr lupus-associated, phenotype in mice is associated mainly with childhood onset SLE only (FIG. 1). Of the 12 SNPs significantly associated with SLE only one shows association in adults as well. The fact that previous studies have not shown significant association with the TNFRSF6 gene may reflect that most previous studies were done with adult onset SLE.

Taken together, we believe that childhood and adult-onset patients have overlapping but districts set of genes/SNPs associated with SLE. Thus, TNFRSF6 is significantly associated almost exclusively with childhood-onset, while IRAK1 and KLRG1 are associated with fully overlapping sets of SNPs. Furthermore, we are in the unique position that we have sufficient numbers of cases in 4 ethnicities so that we can identify those genetic risk factors that are common to multiple ethnic groups and might obtain information explaining differences in disease prevalence and disease progression in diverse populations.

We have also searched for haplotype blocks significantly associated with SLE. Haploview version 4.0 (14) was used to estimate the linkage disequilibrium (LD) between markers and haplotype structures in different ethnicities. Conditional haplotype analyses were conducted using PLINK. We have conducted haplotype analyses and constructed haplotype blocks for each gene separately for every ethnicity and in children and adults separately. Using Haploview we carried out logistic regression analyses to define the haplotype blocks that most likely contain the causal mutation and define the risk haplotype for each ethnic population in children and adults separately. Some examples show several KLRG1 haplotypes significantly associated with SLE in children and adults.

The data shows that there are two common TNFSF4 haplotype blocks significantly associated with SLE in both adults and children and multiple ethnicities. The genes for which we present data here are very attractive candidates, which is not surprising since they belong to biological pathways that were chosen a priori as potentially involved in the pathogenesis of SLE. The following summarizes what is known regarding the biology of these genes in relation to SLE pathogenesis:

SELP or P-selectin (located on chromosome 1q24) is a transmembrane protein expressed on activated platelets and endothelial cells and serves as an adhesion receptor for neutrophils, monocytes, and T cells (15). The interaction between SELP on endothelial cells and its ligands on T cells is responsible for the migration of these cells into inflamed tissues (15). Importantly, levels of platelet-leukocyte complexes and soluble SELP are significantly elevated in SLE patients (16). Of great interest, expression of both glomerular and interstitial SELP is up-regulated in various forms of proliferative glomerulonephritis (GN), including SLE nephritis (17). A recent publication (18) surprisingly shows that SELP-deficient MRL-lpr mice develop accelerated lethal GN. Although instinctively we would have predicted that SELP is necessary for lupus nephritis development, these results suggests that the involvement of this gene in SLE may be much more complex and it is premature to propose specific hypotheses to its mechanism of involvement.

IRAK1 (Interleukin 1 receptor-associated kinase-1) gene (on chromosome Xq28) is a serine/threonine protein kinase involved in the signaling cascade of the Toll/IL-1 receptor (TIR) family (19). The TIR family comprises the IL-1 receptor subfamily, which recognizes the endogenous proinflammatory cytokines IL-1 and IL-18, and the members of the Toll-like receptor (TLR) subfamily that recognize pathogen-associated molecular patterns. A hallmark of the TIR family is the cytoplasmic TIR domain which serves as a scaffold for a series of protein-protein interactions leading to the activation of a unique and exclusive signaling module consisting of MyD88, IRAK family members, and Tollip. Subsequently, several central signaling pathways of the innate and adaptive immune system are activated in parallel, with the activation of NFκB being the most prominent event of the inflammatory response (19). IRAK1 is considered both the “on-switch”, by linking the receptor complex to the central adapter/activator protein TRAF6, as well as the “off-switch” (36), by its auto-induced removal from the complex (20).

The association of KLRGI (killer cell lectin like receptor 1) gene (mapped at 12p 12) implicates the involvement of NK-cells in the genetic predisposition to SLE. KLRG1 is expressed on NK cells and on subsets of activated T-cells. KLRG1-expressing NK cells show decreased proliferative activity (21). SLE patients, including childhood-onset cases, have quantitative and qualitative alterations in NK cells (22-24). The association of SLE with KLRG1 in our studies, coupled to previous findings that first-degree relatives of SLE patients (24) and healthy monozygotic co-twins of SLE patients (25) display reduced numbers and activity of NK cells, suggests that this latter phenotype might be involved in disease causation rather than simply a consequence of the disease process.

Our original study published in 2007 (7) and the replication studies presented here emphasize a highly significant association between SLE and TNFSF4 (OX40L). An independent study lead by Tim Vyse (26) corroborates and extends our findings regarding the involvement of this gene in the predisposition to SLE. One of our associated SNPs that induces a C/G polymorphism in the promoter of OX40L (p=1.1×10⁴) is predicted to alter the binding site for the c-Myc/Max transcription factor (7). Interaction between OX40 and its ligand is involved in co-stimulation of T and B lymphocytes and in adhesion of T cells to endothelial surfaces. Indeed, immunohistology of renal biopsies from SLE patients with proliferative GN demonstrated an abundance of OX40L in all cases in a unique granular distribution which co-localized with sub-epithelial immune deposits (27). Accordingly, promoter region changes of OX40L could have profound consequences for gene expression with attendant significant ramifications for both disease susceptibility and disease severity.

Fas apoptosis inhibitory molecule (FAIM) (at 3q22.3) was originally characterized (by Dr. Thomas Rothstein's group) as Fas-antagonist in B cells and shown to be up-regulated in B cells resistant to Fas-mediated cell death (28). FAIM is highly conserved in evolution and is broadly expressed in many tissues (29). An alternative splice isoform that includes a longer (by 22 amino acids) molecule (FAIM-L) has been identified that is predominantly expressed in the brain (30) and protects neurons from death receptor-triggered apoptosis (31). The abundantly expressed shorter form of FAIM promotes NFκB activation and in neurons also the Ras-ERK pathway (32). We have established collaboration with Dr. Rothstein (letter of collaboration and biosketch attached) to study the potential role of FAIM in SLE. Recently, FAIM has been shown to enhance CD40-mediated induction of NFκB activation in B cells, and, consequently, FAIM enhances up-regulation of several KB-dependent genes including anti-apoptotic genes such as Bcl-xL, likely accounting for FAIM's anti-apoptotic effect (unpublished personal communication, Dr. Thomas Rothstein, The Feinstein Institute, Manhasset, N.Y.).

Lastly, the TLR8 (Toll-like receptor 8) gene (at Xp22) is activated by ssRNA and initiates innate and adaptive immune responses (33, 34). Relevant to the present work, TLR8 is particularly effective in inducing Th-1 polarizing responses from human monocytes and myeloid dendritic cells (33). Its major mode of action is activation of the NFκB pathway using MyD88 as its sole receptor-proximal adaptor to transduce signals (34). Interestingly, TLR8 agonists induce marked surface expression of CD40 on human mDC (35). Since FAIM also induces CD40 and NFκB activation (see above) it is possible that FAIM and TLR8 act in the same pathway regarding their role in the pathogenesis of SLE.

Since several of our candidate genes (IRAK1, TNFRSF6, TLR8 and FAIM) are involved in the NFκB inflammatory pathway (32, 34, 36, 41) are good candidates for testing the hypothesis that these genes may act synergistically to generate and/or enhance the phenotype.

The studies presented demonstrate the powerful potential of using combination of up-to-date biotechnology and bioinformatic methods for discovering novel genes. The extensive involvement of these candidate genes in regulation of immune response makes their association with SLE potentially very important and justifies subsequent genetic and functional studies.

Materials and Methods

High throughput sequencing: The major bottleneck in the next generation sequencing approaches is in isolating and enriching the target DNA prior to sequencing (42). The most common current approach for target DNA isolation includes short PCR and long PCR in which primer pairs complementary to specific genomic regions of interest are used for PCR (43). PCR amplification of the regions of interests is performed using a series of overlapping (˜100-200 by overlap) amplicons from 5-10 kb in length. Actual sizes of amplicons will be determined by the location of identification according to repetitive elements, the position of known polymorphisms, and the ability to design a primer pair given these sequence constraints and optimal primer design (melting temperature, primer-dimer, hairpin formation, etc.). The UCSC genome browser, dbSNP (http://www.ncbi.nlm.nih.gov/SNP/), is used and Primer3 is used to assist with primer design. PCR amplification of large regions can require multiple rounds of optimization and redesign of primers. However, development of improved Taq Polymerase enzyme mixes has resulted in greater design success. Our Core facility have used the SequalPrep Long PCR Kit with dNTP5 (Invitrogen) to successfully amplify 600 kb of genomic with only 40 kb requiring more than 1 round of optimization. Regions that fail the first round of optimization are repeated using a series of alternate melting temperatures (Tm). If needed, a new set of primers may be designed, attempted, and the process repeated until the region is successfully amplified. PCR products are generated using 50 ng of input DNA per amplicon, quantitated, normalized with respect to each other, pooled, and processed through the Genomic DNA preparation protocol (below) to generate a library for DNA sequencing. The following describe the steps involved in next generation sequencing:

a) DNA Library Preparation. DNA libraries are prepared using a Genomic DNA Sample Prep Kit (Illumina, Inc) according to manufacturer's specifications with minor modifications. Fragmented DNA (by sonication) are repaired to blunt ends using a combination of T4 DNA Polymerase and Klenow DNA polymerase. Addition of an ‘A’ base to the 3′ end of blunt DNA is accomplished using Klenow exo (3′->5′ exon minus) and dATP.Adapters with a 3′ ‘T’ overhang are ligated to the end modified DNA. Following adapter ligation, size fractionation of the prepared library is performed using low-range Ultra Agarose gel electrophoresis. Enrichment of adapter modified DNA is accomplished by PCR using primers that anneal to the ends of the adapters.

b) Fragmented DNA sizing. Sizing of fragmented DNA is performed using an Experion automated electrophoresis system and Experion 1K DNA Analysis kit (Bio-Rad).

c) Cluster Formation. Attachment of the adapted DNA to the Illumina flow cell is performed on a Cluster Station using a Cluster Generation kit according to manufacturers instructions. Each flow cell contains 8 1.0 mm lanes with the capability of loading separate samples into each lane. The protocol is typically performed for Amplification/Linearization/Blocking on one day and the flow cell is stored in a 50 ml tube in storage buffer at 4° C. for up to 2 weeks prior to sequencing. Each sample is initially run on a single lane to estimate cluster density. Future lanes are run with a target of 20,000-25,000 clusters per tile. Typically, a linear relationship between cluster density and unique alignment of the sequence reads to the reference genome with up to 20-22,000 clusters per tile is seen. As a control, 1 lane will be dedicated to sequencing of phiX DNA at a cluster density of ˜20,000 clusters per tile. Analysis of the phiX DNA sequences will provide information on the quality of the cluster station and DNA sequencing protocols on a per run basis. In addition, comparison of phiX DNA control sequences over time will serve as a control for run-to-run variation and sequential timepoints of machine and chemistry performance.

d) DNA Sequencing. Prepared flow cells are sequenced on an Illumina Genome Analyzer using a SBS 36-Cycle Sequencing kit (Illumina) according to manufacturer's instructions. Sequencing primer hybridization is performed on the Cluster Station followed by loading the flow cell onto the Genome Analyzer and beginning the first base sequencing. First base metrics is evaluated to determine if the flow cell should be repositioned and if the run should proceed with the current flow cell. If the flow cell metrics do not pass QA/QC, another flow cell is chosen for sequencing primer hybridization and loaded onto the Genome Analyzer as described previously. Our core facility experience is that less than 5% of the flow cells require repositioning and greater than 90% of flow cells pass first base QA/QC. Typically, runs are set to collect 36 cycles of DNA sequencing. Current settings are 300 tiles per lane (3 columns×100 rows per column) and each of the 4 bases are imaged separately per tile. Image data is off-loaded during the run onto a network attached storage device within 45 minutes following each cycle.

Several layers of QA/QC are included during DNA library preparation to reduce possible contamination of the library with non-library and contaminating DNA from other prepared libraries. In addition, QA/QC of the flow cell is performed at 2 stages, the first determines proper seating of the flow cell within the manifold and the second evaluates the cluster intensity within the flow cell.

Alternative approach: As the size and number of regions of interest increase, other approaches have been developed that rely on parallel capture and enrichment (44-46). These higher throughput methods have some advantages over PCR, including use of less input DNA per region, parallel capture of a complex number of regions, less optimization since the entire capture is performed at once, and quicker isolation of captured DNA. However, if the number of target DNA is small, PCR will use less DNA and be more cost effective.

Currently, both Nimblegen and Agilent are providing chip technologies specifically designed for the capture of targeted genomic regions for next generation sequencing. The first two steps in the capture-array based genomic selection method are very similar to the methodology described above; namely, physical shearing of genomic DNA to create random fragments, followed by end repair of the fragments, which includes adding 3′-adenine overhangs, and ligation to unique adaptors with complementary thymine overhangs. The following steps are unique for the capture arrays and include fragment hybridization and capture using a custom high-density oligonucleotide microarray consisting of complementary sequences identified from a reference genome sequence, and elution of fragments bound to the probe. The capture-arrays are ideal for regions of 1 Mb. Our specific situation is a scenario with an estimated 300-400 kb of sequencing. We would like to develop a specific genomic capture chip for our proposed study of selected candidate genes for human SLE. For this application, we target a smaller segment of total genomic DNA but would like to take advantage of capture chip technology together with a newly-developed indexing system of the next generation genomic sequencers in order to reduce our cost and increase throughput.

The indexing system is a strategy that involves the addition of a specific “indexing” sequence into the sequence primers that are ligated to both ends of randomly sheared genomic fragments as the first step in the sequencing technology. The “index” sequence is a short (8-12 bp) specific sequence that is added to the interior end of the primer sequence. These indexing sequence primers allow a specific index sequence to be ligated to individual samples prior to pooling. The sequence of this “indexing” segment will be determined at the initiation of each sequencing reaction, thus allowing the subsequently determined sequence to be assigned to a specific sample, even when fragments derived from several different “indexed” samples are being analyzed in the same sequencing run. These “indexing” primers will allow 10 or more DNA samples to be indexed, pooled and sequenced in a single run. The resultant sequences can then be assigned to the data output for each sample during the sequence analysis phase.

Step 3 will commence after identifying new variants within the haplotype block of interest. In this part we will re-evaluate these new SNP in the entire available study population in a case control association study using the different ethnicities and both childhood- and adult-onset cohorts similarly to our preliminary studies. All technical and analytical procedures to be used here have been described in section 3 above.

It is expected that among potentially functional variants, the causal SNP (and/or SNPs in complete LD) should exhibit the strongest evidence for association within the haplotype block.

TABLE 1
Genes associated with SLE according to TDT and q value analysis.

| | | | | | Amino | Amino | | | | |
|---|---|---|---|---|---|---|---|---|---|---|
| | gene | gene | SNP | | acid | acid | | | | accession |
| SNP ID | symbol | location | location | Alleles | change | position | p | TDT | Q value | number |
| rs3917815 | SELP | 1q24.2 | Coding | A/G | N/S | 673 | 5.74 × 10⁻⁶ | 20.571 | 0.025 | NP_002996 |
| | | | exon | | | | | | | |
| rs10127175 | IRAK1 | Xq28 | Coding | A/T | C/S | 203 | 9.58 × 10⁻⁵ | 19.593 | 0.028 | NP_001020413 |
| | | | exon | | | | | | | |
| rs1805749 | KLRG1 | 12p13.31 | Coding | A/G | W/R | 58 | 8.77 × 10⁻⁵ | 15.385 | 0.153 | NP_005801 |
| | | | exon | | | | | | | |
| rs2274065 | NCF2 | 1q25.3 | Intron | A/C | — | — | 7.28 × 10⁻⁵ | 15.736 | 0.153 | NP_000424 |
| rs1234314 | TNFSF4 | 1q25.1 | Promoter | C/G | — | — | 1.14 × 10⁻⁴ | 14.885 | 0.166 | NP_003317 |
| rs4728142 | IRF5 | 7q32.1 | Promoter | A/G | — | — | 1.92 × 10⁻⁴ | 13.909 | 0.239 | NP_002191 |
| rs6072794 | PTPRT | 20q12 | Intron | C/T | — | — | 2.65 × 10⁻⁴ | 13.302 | 0.257 | NP_008981 |
| rs10406301 | KIR2DS4 | 19q13.42 | Coding | C/G | S/C | 103* | 2.56 × 10⁻⁴ | 13.370 | 0.257 | NP_036446 |
| | | | exon | | | | | | | |
| rs4406737 | TNFRSF6 | 10q23.31 | Intron | A/G | — | — | 3.78 × 10⁻⁴ | 12.636 | 0.330 | NP_000034 |

*a splicing isoform has the substitution at position 96 (accession no. NP_839942).

TABLE 2
Representative SNPs associated with SLE.

| | | | | | Adult | | | | | Childhood | | | |
|---|---|---|---|---|---|---|---|---|---|---|---|---|---|
| gene | | Comb. | Comb. | | Freq. | Freq. | | | | Freq. | | | |
| name | SNP | q Adult | q Child | Ethnic. | Control | Case | OR | p | q | Case | OR | p | q |
| FAIM | rs13095734 | 5.96E−3 | 3.78E−3 | AA | 0.95 | 0.89 | 0.46 | 2.17E−10 | 7.68E−3 | 0.90 | 0.51 | 8.03E−3 | 2.87E−2 |
| | | | | EA | 0.92 | 0.90 | 0.79 | 3.75E−4 | 7.73E−3 | 0.88 | 0.61 | 8.96E−3 | 3.09E−2 |
| | | | | MA | 0.66 | 0.82 | 2.36 | 8.70E−14 | 7.68E−3 | 0.92 | 5.96 | 5.09E−26 | 9.89E−3 |
| | | | | Asian | 0.42 | 0.50 | 1.39 | 9.35E−6 | 7.68E−3 | 0.61 | 2.15 | 1.54E−10 | 9.89E−3 |
| Fas | rs3758485 | 1.66E−1 | 3.78E−3 | AA | 0.21 | 0.19 | 0.89 | 1.57E−1 | 2.31E−1 | 0.19 | 0.90 | 5.16E−1 | 4.06E−1 |
| | | | | EA | 0.39 | 0.40 | 1.03 | 4.13E−1 | 3.85E−1 | 0.37 | 0.90 | 3.15E−1 | 3.07E−1 |
| | | | | MA | 0.37 | 0.36 | 0.95 | 6.19E−1 | 4.76E−1 | 0.31 | 0.76 | 4.99E−2 | 1.00E−1 |
| | | | | Asian | 0.53 | 0.52 | 0.93 | 2.73E−1 | 3.11E−1 | 0.41 | 0.60 | 7.18E−5 | 9.89E−3 |
| | rs2234978 | 4.12E−2 | 3.78E−3 | AA | 0.68 | 0.69 | 1.04 | 5.48E−1 | 4.49E−1 | 0.66 | 0.93 | 5.86E−1 | 4.33E−1 |
| | | | | EA | 0.71 | 0.71 | 1.00 | 9.29E−1 | 5.78E−1 | 0.68 | 0.90 | 3.65E−1 | 3.34E−1 |
| | | | | MA | 0.77 | 0.83 | 1.39 | 1.38E−2 | 4.21E−2 | 0.83 | 1.41 | 2.93E−2 | 7.09E−2 |
| | | | | Asian | 0.98 | 0.97 | 0.65 | 6.62E−2 | 1.33E−1 | 0.93 | 0.28 | 9.71E−6 | 9.89E−3 |
| IRAK1 | rs763737 | 5.96E−3 | 3.78E−3 | AA | 0.46 | 0.44 | 0.94 | 3.52E−1 | 3.59E−1 | 0.44 | 0.95 | 7.01E−1 | 4.79E−1 |
| | | | | EA | 0.21 | 0.23 | 1.11 | 3.89E−2 | 9.07E−2 | 0.29 | 1.52 | 2.50E−3 | 1.42E−2 |
| | | | | MA | 0.49 | 0.55 | 1.28 | 3.05E−2 | 7.69E−2 | 0.62 | 1.69 | 3.31E−4 | 9.89E−3 |
| | | | | Asian | 0.74 | 0.83 | 1.63 | 5.13E−8 | 7.68E−3 | 0.82 | 1.57 | 5.93E−3 | 2.33E−2 |
| | rs5945174 | 5.96E−3 | 5.45E−3 | AA | 0.32 | 0.34 | 1.11 | 1.56E−1 | 2.31E−1 | 0.32 | 0.98 | 9.07E−1 | 5.45E−1 |
| | | | | EA | 0.14 | 0.18 | 1.34 | 6.76E−8 | 7.68E−3 | 0.19 | 1.40 | 2.60E−2 | 6.63E−2 |
| | | | | MA | 0.34 | 0.43 | 1.46 | 1.16E−3 | 9.30E−3 | 0.39 | 1.23 | 1.03E−1 | 1.61E−1 |
| | | | | Asian | 0.48 | 0.50 | 1.09 | 1.61E−2 | 4.69E−2 | 0.43 | 0.82 | 1.23E−2 | 3.84E−2 |
| KLRG1 | rs10400563 | 2.46E−2 | 8.13E−3 | AA | 0.65 | 0.62 | 0.86 | 2.37E−2 | 6.32E−2 | 0.58 | 0.73 | 1.35E−2 | 4.09E−2 |
| | | | | EA | 0.75 | 0.75 | 0.99 | 7.95E−1 | 5.34E−1 | 0.71 | 0.84 | 1.13E−1 | 1.70E−1 |
| | | | | MA | 0.76 | 0.74 | 0.94 | 6.21E−1 | 4.76E−1 | 0.75 | 0.95 | 7.24E−1 | 4.85E−1 |
| | | | | Asian | 0.50 | 0.46 | 0.85 | 1.45E−2 | 4.29E−2 | 0.44 | 0.80 | 6.89E−2 | 1.23E−1 |
| | rs11048434 | 5.96E−3 | 5.11E−3 | AA | 0.86 | 0.90 | 1.44 | 2.74E−4 | 7.68E−3 | 0.90 | 1.47 | 7.84E−2 | 1.33E−1 |
| | | | | EA | 0.64 | 0.63 | 0.99 | 3.71E−1 | 5.60E−1 | 0.67 | 1.15 | 1.84E−1 | 2.27E−1 |
| | | | | MA | 0.67 | 0.63 | 0.86 | 1.81E−1 | 2.49E−1 | 0.63 | 0.87 | 2.99E−1 | 3.01E−1 |
| | | | | Asian | 0.57 | 0.63 | 1.28 | 3.47E−4 | 7.73E−3 | 0.65 | 1.42 | 5.85E−3 | 2.32E−2 |
| SELP | rs3917843 | 5.96E−3 | 6.35E−2 | AA | 0.03 | 0.01 | 0.53 | 6.89E−3 | 2.42E−2 | 0.02 | 0.62 | 9.19E−1 | 5.50E−1 |
| | | | | EA | 0.08 | 0.05 | 0.74 | 2.16E−4 | 7.68E−3 | 0.08 | 1.23 | 3.12E−1 | 3.07E−1 |
| | | | | MA | 0.12 | 0.13 | 1.09 | 6.00E−1 | 4.66E−1 | 0.15 | 1.30 | 1.76E−1 | 2.22E−1 |
| | | | | Asian | 0.61 | 0.61 | 0.99 | 8.50E−1 | 5.55E−1 | 0.56 | 0.82 | 1.17E−1 | 1.73E−1 |
| | rs2205895 | 9.54E−2 | 3.78E−3 | AA | 0.68 | 0.70 | 1.12 | 8.95E−2 | 1.63E−1 | 0.66 | 0.94 | 6.49E−1 | 4.57E−1 |
| | | | | EA | 0.64 | 0.64 | 0.99 | 8.59E−1 | 5.58E−1 | 0.58 | 0.75 | 7.04E−3 | 2.62E−2 |
| | | | | MA | 0.46 | 0.43 | 0.88 | 2.41E−1 | 2.93E−1 | 0.40 | 0.79 | 7.30E−2 | 1.27E−1 |
| | | | | Asian | 0.04 | 0.05 | 1.31 | 1.38E−1 | 2.17E−1 | 0.11 | 3.19 | 8.42E−6 | 9.89E−3 |
| TLR8 | rs176995 | 2.87E−1 | 3.78E−3 | AA | 0.70 | 0.71 | 1.07 | 3.99E−1 | 3.83E−1 | 0.72 | 1.13 | 4.22E−1 | 3.58E−1 |
| | | | | EA | 0.84 | 0.85 | 1.04 | 4.55E−1 | 4.04E−1 | 0.80 | 0.74 | 4.28E−2 | 9.23E−2 |
| | | | | MA | 0.87 | 0.88 | 1.08 | 6.52E−1 | 4.83E−1 | 0.88 | 1.10 | 6.34E−1 | 4.51E−1 |
| | | | | Asian | 0.99 | 0.99 | 0.82 | 6.97E−1 | 5.00E−1 | 0.95 | 0.11 | 4.98E−5 | 9.89E−3 |
| | rs17258081 | 5.96E−3 | 3.78E−3 | AA | 0.80 | 0.77 | 0.84 | 5.22E−2 | 1.11E−1 | 0.72 | 0.64 | 1.94E−2 | 5.25E−2 |
| | | | | EA | 0.61 | 0.58 | 0.89 | 6.76E−3 | 2.40E−2 | 0.58 | 0.88 | 3.41E−1 | 3.22E−1 |
| | | | | MA | 0.75 | 0.61 | 0.53 | 7.35E−6 | 7.68E−3 | 0.59 | 0.50 | 7.63E−5 | 9.89E−3 |
| | | | | Asian | 0.88 | 0.88 | 1.02 | 8.58E−1 | 5.58E−1 | 0.78 | 0.50 | 2.63E−4 | 9.89E−3 |
| TNFSF4 | rs1234315 | 5.96E−3 | 3.78E−3 | AA | 0.78 | 0.80 | 1.16 | 5.61E−2 | 1.17E−1 | 0.83 | 1.37 | 5.42E−2 | 1.04E−1 |
| | | | | EA | 0.47 | 0.51 | 1.17 | 1.71E−5 | 7.68E−3 | 0.54 | 1.31 | 7.68E−3 | 2.79E−2 |
| | | | | MA | 0.54 | 0.64 | 1.52 | 7.81E−5 | 7.68E−3 | 0.67 | 1.74 | 1.71E−5 | 9.89E−3 |
| | | | | Asian | 0.36 | 0.45 | 1.47 | 4.54E−8 | 7.68E−3 | 0.49 | 1.75 | 9.96E−6 | 9.89E−3 |

### REFERENCES

1. Russ V, Hochberg M C (2002). In: Wallace D J, Hahn B H (eds) Dubois' Lupus Erythematosus, Lippincott Williams & Wilkins, Philadelphia, pp 65-83.
2. Tsao B P (2004) Update on human systemic lupus erythematosus genetics. Curr Opin Rheumatol. 16:513-521.
3. Kyogoku C, Langefeld C D, Ortmann W A, Lee A, Selby S, Carlton V E, Chang M, Ramos P, Baechler E C, Batliwalla F M, Novitzke J, Williams A H, Gillett C, Rodine P, Graham R R, Ardlie K G, Gaffney P M, Moser K L, Petri M, Begovich A B, Gregersen P K, Behrens T W (2004) Genetic association of the R620W polymorphism of protein tyrosine phosphatase PTPN22 with human SLE. Am J Hum Genet. 75:504-507.
4. Ardle K G, Kruglyak L, Seieslstad M. (2002) Patterns of linkage disequilibrium in human genome. Nat Rev Genet 3:299-309.
5. Colhoun H M, McKeigue P M, Davey Smith G (2003) Problems of reporting genetic associations with complex outcomes. Lancet 361:865-872.
6. Freimer N, Sabatti C (2004) The use of pedigree, sib-pair and association studies of common diseases for genetic mapping and epidemiology. Nat Genet 36:1045-1051.
7. Jacob C O, Reiff A, Armstrong D L, Myones B L, Silverman E, Klein-Gitelman M, McCurdie D, Wagner-Weiner L, Nocton J J, Solomon A and Zidovetzki R: Identification of novel susceptibility genes in childhood-onset SLE using a uniquely designed candidate gene pathway platform. Arthritis & Rheum 56:4164-4173, 2007
8. Cassidy J T. in Textbook of Pediatric Rheumatology. eds Cassidy J T, Petty R E. (Elsevier Saunders, Philadelphia), 1996; pp. 329-406.
9. Lehman T J A. in Dubois' Lupus Erythematosus, eds. Wallace D J, Hahn B H, (Lippincott Williams & Wilkins, Philadelphia), 2002; pp 863-884.
10. Spielman R S, McGinnis R E, Ewens W J (1993) Transmission test for linkage disequilibrium: the insulin gene region and insulin-dependent diabetes mellitus (IDDM). Am J Hum Genet 52:506-516.
11. Storey J D, Tibshirani R (2003) Statistical significance for genomewide studies. Proc Nati Acad Sci USA 100:9440-9445.
12. Wacholder S, Chanock S, Garcia-Closas M, El Ghormli L, Rothman N (2004) Assessing the probability that a positive report is false: an approach for molecular epidemiology studies. J Natl Cancer Inst 96:434-442.
13. Purcell S, Neale B, Todd-Brown K, Thomas L, Ferreira M A R, Bender D, Mailer J, Sklar P, de Bakker P I W, Daly M J & Sham P C (2007) PLINK: a toolset for whole-genome association and population-based linkage analysis. American Journal of Human Genetics, 81.
14. Barrett J C, Fry B, Mailer J, Daly M J. (2005) Haploview: analysis and visualization of LD and haplotype maps. Bioinformatics. 21:263-5.
15. Ley K. The role of selectins in inflammation and disease. Trends Mol Med 2003; 9:263-268.
16. Joseph J E, Harrison P, Mackie I J, Isenberg D A, Machin S J. Increased circulating platelet-leucocyte complexes and platelet activation in patients with antiphospholipid syndrome, systemic lupus erythematosus and rheumatoid arthritis. Br J Haematol 2001; 115:451-459.
17. Segawa C, Wada T, Takaeda M, Furuichi K, Matsuda I, Hisada Y, Ohta S, Takasawa K, Takeda S, Kobayashi K, Yokoyama H. In situ expression and soluble form of P-selectin in human glomerulonephritis. Kidney Int 1997; 52:1054-1063.
18. He X, Schoeb T R, Panoskaltsis-Mortari A, Zinn K R, Kesterson R A, Zhang J, Samuel S, Hicks M J, Hickey M J, Bullard D C. Deficiency of P-selectin or P-selectin glycoprotein ligand-1 leads to accelerated development of glomerulonephritis and increased expression of CC chemokine ligand 2 in lupus-prone mice. J. Immunol. 2006; 177:8748-8756.
19. Martin M U, Wesche H. Summary and comparison of the signaling mechanisms of the Toll/interleukin-1 receptor family. Biochim Biophys Acta. 2002; 1592:265-280.
20. Kollewe C, Mackensen A C, Neumann D, Knop J, Cao P, Li S, Wesche H, Martin M U. Sequential autophosphorylation steps in the interleukin-1 receptor-associated kinase-1 regulate its availability as an adapter in interleukin-1 signaling. J Biol Chem 2004; 279:5227-5236.
21. Voehringer D, Koschella M, Pircher H. Lack of proliferative capacity of human effector and memory T cells expressing killer cell lectinlike receptor G1 (KLRG1). Blood 2002; 100:3698-3702.
22. Erkeller-Yusel F, Hulstaart F, Hannet I, Isenberg D, Lydyard P. Lymphocyte subsets in a large cohort of patients with systemic lupus erythematosus. Lupus 1999; 2:227-231.
23. Yabuhara A, Yang F C, Nakazawa T, Iwasaki Y, Mori T, Koike K, Kawai H, Komiyama A. A killing defect of natural killer cells as an underlying immunologic abnormality in childhood systemic lupus erythematosus. J Rheumatol 1996; 23:171-177.
24. Green M R, Kennell A S, Larche M J, Seifert M H, Isenberg D A, Salaman M R. Natural killer cell activity in families of patients with systemic lupus erythematosus: demonstration of a killing defect in patients. Clin Exp Immunol. 2005; 141:165-173.
25. Stohl W, Elliott J E, Hamilton A S, Deapen D M, Mack T M, Horwitz D A. Impaired recovery and cytolytic function of CD56+ T and non-T cells in systemic lupus erythematosus following in vitro polyclonal T cell stimulation. Studies in unselected patients and monozygotic disease-discordant twins. Arthritis Rheum 1996; 39:1840-1851.
26. Graham D S, Graham R R, Manku H, Wong A K, Whittaker J C, Gaffney P M, Moser K L, Rioux J D, Altshuler D, Behrens T W, Vyse T J. Polymorphism at the TNF superfamily gene TNFSF4 confers susceptibility to systemic lupus erythematosus. Nat Genet 2008 40:83-9, 2008
27. Aten J, Roos A, Claessen N, Schilder-Tol E J, Ten Berge I J, Weening J J. Strong and selective glomerular localization of CD134 ligand and TNF receptor-1 in proliferative lupus nephritis. J Am Soc Nephrol 2000; 11:1426-1438.
28. Schneider T J, Fischer G M, Donohoe T J, Colarusso T P, Rothstein T L. A novel gene coding for a Fas apoptosis inhibitory molecule (FAIM) isolated from inducibly Fas-resistant B lymphocytes. J Exp Med. 1999 189:949-56.
29. Rothstein, T. L., X. Zhong, B. R. Schram, R. S. Negm, T. J. Donohoe, D. S. Cabral, L. C. Foote, and T. J. Schneider. 2000. Receptor-specific regulation of B cell susceptibility to Fas-mediated apoptosis and a novel Fas apoptosis inhibitory molecule (FAIM). Immunol. Rev. 176:116.
30. Zhong X, Schneider T J, Cabral D S, Donohoe T J, Rothstein T L. An alternatively spliced long form of Fas apoptosis inhibitory molecule (FAIM) with tissue-specific expression in the brain. Mol Immunol. 2001 38:65-72.
31. Segura M F, Sole C, Pascual M, Moubarak R S, Perez-Garcia M J, Gozzelino R, Iglesias V, Badiola N, Bayascas J R, Llecha N, Rodriguez-Alvarez J, Soriano E, Yuste V J, Comella J X. The long form of Fas apoptotic inhibitory molecule is expressed specifically in neurons and protects them against death receptor-triggered apoptosis. J. Neurosci. 2007 27:11228-41.
32. Sole C, Dolcet X, Segura M F, Gutierrez H, Diaz-Meco M T, Gozzelino R, Sanchis D, Bayascas J R, Gallego C, Moscat J, Davies A M, Comella J X. The death receptor antagonist FAIM promotes neurite outgrowth by a mechanism that depends on ERK and NF-kapp B signaling. J Cell Biol. 2004. 167:479-92.
33. Philbin V J, Levy O. Immunostimulatory activity of TLR8 agonists towards human leukocytes: basic mechanisms and translational opportunities. Biochem. Soc. Trans. 2007. 35:1485-1491.
34. Wang R-F, Miyahara Y, Wang H Y. TLRs and immune regulation. Oncogene 2008. 27:181-189.
35. Schon M P, Schon M. TLR7 and TLR8 as targets in cancer therapy. Oncogene 2008. 27:190-199.
36. Gottipati S, Rao N L, W-P Fung-Leung. IRAK1: A critical signaling mediator of innate immunity. Cell. Sign. 2008, 20:269-276.
37. Sawalha A H, Webb R, Han S, Kelly J A, Kaufman K M, Kimberly R P, Alarcon-Riquelme M E, James J A, Vyse T J, Gilkeson G S, Choi C B, Scofield R H, Bae S C, Nath S K, Harley J B. Common Variants within MECP2 Confer Risk of Systemic Lupus Erythematosus. PloS ONE. 2008 Mar. 5; 3(3):e1727.
38. Sawalha A H, Richardson B C (2005) DNA methylation in the pathogenesis of systemic lupus erythematosus. Current Pharmacogenomics 3: 73-78.
39. McVean G A, Myers S R, Hunt S, Deloukas P, Benteley D R, Donnelly P. The fine-scale structure of recombination rate variation in the human genome. Science. 2004, 304:581-584.
40. Glatt C E, DeYoung J A, Delgado S, Service S K,. Giacomini K M, Robert H. Edwards R H, Risch N, Freimer N B. Screening a large reference sample to identify very low frequency sequence variants: comparisons between two genes. Nature Gen 2001; 27:435-438
41. Kreuz S, Siegmund D, Rumpf J J, Samel D, Leverkus M, Janssen O, Hacker G, Dittrich-Breiholz O, Kracht M, Scheurich P, Wajant H. NFkappaB activation by Fas is mediated through FADD, caspase-8, and RIP and is inhibited by FLIP. J Cell Biol. 2004; 166:369-380
42. Okou D T, Meltz Steinberg K, Middle C, Cutler D J, Albert T J, Zwick M E. Microarray-based genomic selection for high-throughput resequencing. Nat Methods 2007 4: 907-909
43. Sjoblom, T. et al. The Consensus Coding Sequences of Human Breast and Colorectal Cancers Science 2006; 314, 268-274.
44. Gregory J Porreca et al. Multiplex amplification of large sets of human exons. Nat Methods 2007; 4:931-936
45. Albert, T. J. et al. Direct selection of human genomic loci by microarray hybridization Nat. Methods 2007, 4:903-905.
46. Hodges E et al., Genome-wide in situ exon capture for selective resequencing Nat Gen 2007, 39:1522-1527.

### Example 2

Systemic lupus erythematosus (SLE) is a debilitating multisystem autoimmune disorder affecting 0.1% of the North American population (predominantly females). It is characterized by chronic inflammation in various organ systems such as the skin, joints, kidneys, lungs, and brain and the production of autoantibodies to multiple self antigens ([1]). Genome-wide linkage studies have been performed in small to medium-sized collections of families with 2 or more affected members, and several genetic intervals have been identified ([2-7]), some of them corroborated in 2 or more independent studies ([8-11]). Taken together, the findings of these studies suggest that multiple genes contribute to the pathogenesis of SLE, each providing quite modest genetic effects. Furthermore, these studies have shown that the genetics of SLE are not dominated by a single major genetic effect (such as the effect of HLA in type 1 diabetes mellitus or rheumatoid arthritis, both autoimmune diseases).

While the linkage analysis methods used to date have been quite successful in identifying rare variants with strong genetic effects, this approach has limited power to detect common variants with more modest effects. Although some rare alleles with strong genetic effects (such as C1q deficiency) can contribute to SLE genetics, it is probable that common alleles with modest genetic effects play a more important role in disease susceptibility. Thus, we hypothesized that many genetic alleles important to the SLE phenotype will not be identified through genome-wide linkage studies. As a case in point, it was recently shown that an allele of PTPN22 (the gene for protein tyrosine phosphatase N22, a lymphocytic phosphatase that is capable of decreasing T cell activation) is a risk factor for SLE, with an odds ratio of 4 ([12]). PTPN22 is encoded on chromosome 1 at 1p12, a region that was not identified in any of the SLE linkage studies.

Since association studies are more powerful than linkage studies when the predisposing variant is more frequent and when the genes have a moderate association with the disease ([13][14]), a better strategy would be to perform a series of candidate gene single-nucleotide polymorphism (SNP) screens in a study population that is most conducive to expression of these susceptibility genes. However, it is becoming increasingly clear that association studies performed with a wide, random selection of candidate genes are unlikely to yield reproducible results; indeed, it has been estimated that the rate of false-positive results in such studies is near 95% ([15]). Accordingly, it has been suggested that a Bayesian methodology (wherein instead of selecting candidate genes at random, the investigators select the candidate genes based on prior available information) is one way to increase the reliability of association studies and to increase the likelihood of finding genes actually associated with a disease ([15][16]).

In this report we describe a novel strategy using a combination of state-of-the-art hardware and analysis methods to investigate genetics of complex diseases, whereby the investigation is initiated with a bioinformatics-driven design of a custom-made chip that incorporates close to 10,000 SNPs derived from 1,000 selected genes. This chip was used to genotype families with childhood-onset SLE, and data were analyzed using rigorous statistical methods including multicomparison correction.

### Patients and Methods

The University of Southern California Institutional Review Board for research on human subjects approved this study. The study was also approved by the Human Subject Institutional Review Boards at each institution from which subjects were recruited, and informed consent was obtained from all subjects (parents provided consent on behalf of children who were under the legal age of consent).

Inclusion criteria and data collection. For the purposes of this study we considered a subject to have childhood-onset SLE if the American College of Rheumatology (ACR) criteria for SLE ([17][18]) were fulfilled and the diagnosis of SLE was made before the subject was 13 years old, by at least 1 pediatric rheumatologist participating in the study. Each SLE patient and his/her parents were interviewed, and a family history was obtained. We collected data describing a fixed family structure (proband's grandparents, parents, and siblings). In addition to self-declared ethnicity, information on the birthplace of the subject, the parents, and the grandparents was collected, for accurate ethnic characterization of families. For each case, information regarding sex, date of birth, date of first symptoms, and date of diagnosis was collected. For all cases, medical records documenting SLE diagnosis and disease progression, including all treatments and results of all serologic and chemical blood tests, biopsies, and radiologic studies, were reviewed by at least 1 pediatric rheumatologist. When possible, disease severity was evaluated, based on the number of organs involved and severity of involvement, using the Systemic Lupus International Collaborative Clinics/ACR Damage Index ([19]). All of this information was collected and imported into our database. Blood was collected and genomic DNA and plasma prepared and stored according to standard procedures.

Subjects. The 753 subjects in the present study (representing 251 complete trio families) were a subsample of those in the University of Southern California Childhood-Onset SLE Genetics Study database, projected to reach 850 childhood-onset SLE cases by the end of 2008. In parallel, 536 adult-onset SLE patients and their families have been recruited from the same populations and geographic areas (51).

DNA preparation. Blood samples were collected from all participants, and genomic DNA was extracted from peripheral blood mononuclear cells by standard procedures. Resultant DNA, resuspended in Tris-EDTA buffer, was quantified initially using an ND-1000 spectrophotometer (NanoDrop Technologies, Wilmington, Del.). Before genotyping, DNA was requantified using PicoGreen reagent. Samples were normalized to a concentration of 150 ng/μl and interdigitated into 96-well plates.

Genotyping. Molecular inversion probes were designed and produced at ParAllele Biosciences (Palo Alto, Calif.) and printed on Affymetrix GeneChip Tag arrays. Genotyping reactions were carried out according to the manufacturer's recommendations, using previously described protocols ([20][21]). Molecular inversion assays were performed with the MegAllele genotyping kit (ParAllele Biosciences), with 96-well plates and samples from 24 subjects per plate for each of 4 allele channels. Genotypes were scored at ParAllele Biosciences, using Euclidian clustering analysis of the contrast measures derived from the normalized signal intensities. Relative intensities of 2 expected allele bases and 2 background bases indicate genotype and probe performance.

Statistical analysis. The transmission disequilibrium test (TDT) ([22]) was used to evaluate transmission disequilibrium between the 2 alleles at each of the SNPs. The TDT statistics are calculated from the ratio of transmission of an allele to an affected child from a heterozygous parent, or TDT=(b−c)2/(b+c), where b is the number of transmissions of the first allele to affected children from a heterozygous parent and c is the number of transmissions of the second allele to affected children from a heterozygous parent. In order to calculate TDT statistics, we used a custom Perl program which calculated the TDT for each SNP. The calculation was done with concomitant reductions in memory usage over equivalent programs which read the entire data file; we tested the output of our custom TDT program at selected SNPs, and found it to be identical to the output generated with Spielman's TDT/S-TDT suite (http://genomics.med.upenn.edu/spielman/TDT.htm). The TDT statistics have a chi-square distribution with 1 df; we used R (http://www.R-project.org) to calculate P values from the TDT statistics. To correct for multiple hypothesis testing, we applied the q value correction, derived from the false discovery rate (FDR) ([23]), to the resultant P values, using the qvalues package for R (http://cran.r-project.org/src/contrib/Descriptions/qvalue.html).

False-positive report probability (FPRP) was estimated as described by Wacholder et al ([24]), i.e., FPRP=1/{1+Π[/(1−Π)][(1−β)/p]}, where Π is the prior Bayesian probability of the alternative hypothesis being true, (1−β) is the power of the TDT, calculated using the significance level=0.05 (corresponding to X_{\acute{α}}=1.6), and p is taken from the TDT P values. This is a slightly different representation (although the same formalism) from the original approach, whereby we were asking the question, If we set as a significant probability alpha the P value from the TDT test, what corresponding limiting value of FPRP will be obtained?

Transcription factor binding site analysis. The search for transcription factor binding sites on allele variants with the SNPs located in the promoter regions was performed using the TRANSFAC database, accessed via Match interface (http://www.gene-regulation.com/cgi-bin/pub/programs/match/) ([25]).

### Results

Platform design, microchip production, and validation. In the present study we adopted an essentially Bayesian approach, but rather than concentrating on specific candidate genes, we developed a collection of candidate pathways. To this end, we took advantage of the accumulated data from genome-wide scans of adult SLE families, candidate gene investigations, genetic information gained from studies of mouse models of lupus, and gene expression profiling data on human SLE. Based on our examination of the literature, we selected a list of candidate functional pathways judged to be relevant to the pathogenesis of SLE. Three databases (NCBI [http://www.ncbi.nlm.nih.gov/SNPI], Gene Cards [http://www.genecards.org/], and Harvester [http://harvester.embl.de/]) were searched using a set of key words representing these functional pathways (51).

This initial inclusive search resulted in the selection of 6,384 genes. Subsequent analyses were conducted to identify the following: 1) genes that could be excluded based on their expression pattern or function in unrelated processes (e.g., believed to be involved only in embryogenesis or expressed in tissues deemed irrelevant), 2) genes initially included solely on the basis of their homology to a relevant gene, and 3) genes without any known or predicted function. Genes in the latter 2 groups were included for further analysis only if they resided within established linkage peaks. Finally, the number of times a gene was picked up using distinct key words was scored and utilized in prioritization (pathway score [see below]). Using the above criteria, a final list of 1,204 genes was selected (51).

The choice of SNPs within the selected genes was based on available information from databases and accumulating information from the Human Haplotype Mapping Project (HapMap). Priority was given to SNPs demonstrating high heterozygosity, those that were informative in 2 or more relevant ethnicities, and those representing amino acid coding variants. The list of SNPs was then cross-checked against the accumulated SNP validation test results available through ParAllele Biosciences, an active participant in the International HapMap project. A final list of 9,412 SNPs was selected for genotyping assay development. To this end, ParAllele molecular inversion probe technology on the Affymetrix TAG3 platform was used ([21]). The molecular inversion probe assay relies on enzymatic specificity, rather than the hybridization specificity of other chip-based approaches. Enzymatic specificity is sensitive to single base changes, thereby reducing false-positive signal. In addition, the insensitivity of these inversion probes to intermolecular interactions allows the probes to be multiplexed so that all 9,412 SNPs could be genotyped in a single assay. The genotyping platform was validated on 18 control samples and 5 complete HapMap trios. Of the 9,412 SNPs, robust genotyping data were generated for 9,375 (99.6%). Several control samples were genotyped up to 8 times, resulting in 99.98% reproducibility of these genotypes.

Characteristics of the SLE patients. The candidate pathway genotyping platform developed as described above was applied to a sample of 753 subjects, corresponding to 251 childhood-onset SLE trios (patients and both of their parents). Since kidney involvement is one of the most devastating complications of SLE, it is noteworthy that 60% of our patients with childhood-onset SLE had kidney disease, whereas kidney disease occurred in fewer than one-third of the adult-onset SLE patients (51). Similarly, while 20% of the adults with SLE had cardiac or pulmonary involvement, this complication was present in >50% of our childhood-onset patients. Childhood SLE is more often a multisystem disease than is adult-onset SLE, as was further exemplified by the fact that 29% of the childhood-onset SLE patients manifested a neurologic disorder, while it appeared in <10% of the adults with SLE. Nevertheless, childhood- and adult-onset SLE exhibited similar sets of manifestations, albeit at different frequencies, and responded to similar therapies, supporting the notion that they are the same disease. Because sex hormones are less likely to play an important role in the onset of disease in children, a much higher frequency of males was found in our childhood-onset cohort compared with the adult-onset cohort (38% versus 9%), and the female:male ratio was reduced from 9:1 in the adult-onset group to 3:2 in the childhood-onset group (51).

Genes identified by family-based TDT. TDT ([22]) was used to calculate the significance of SNP association with SLE. A confounding effect due to population stratification was avoided by using the family-based TDT, in which the preferential transmission of the test allele from parents to affected offspring provides evidence of association of the test allele with disease.

The standards of statistical proof that are commonly used in biomedical literature have been questioned when applied to large SNP-based genetic association studies. The problem of multiple testing pervades the discipline, without a clear consensus on how it should be solved ([26]). The classic Bonferroni correction is both too strict and inappropriate in the case of genetic studies because it assumes that each test is independent, whereas in actuality a complex and unknown mutual dependence is present among genes, and even more prominently among SNPs of the same gene. The FDR approach ([27]) is currently widely used in genetic microarray and association studies. We adapted a variation of the FDR ([23]) for the multitest correction in our study.

We decided on 2 levels of FDR as representing significant outcomes in this study: SNPs with q values of <0.05 would be considered as proven with >95% probability, and those with q values of <0.5 as noteworthy and requiring followup studies for verification. 2 genes, SELP (gene for P-selectin) and IRAK1 (gene for interleukin-1 receptor-associated kinase 1 [IRAK-1]) fell into the first category (51). Indeed, the most significant associations found in the present study were with a polymorphism at amino acid position N673S in SELP (X²=20.571, P=5.74×10−6) and with a polymorphism at amino acid position C203S in IRAK1 (X²=19.593, P=9.58×10⁻⁶). The N673S polymorphism in P-selectin is located in the eighth Sushi domain of the protein. Sushi domains (complement control protein modules) are characteristic of a variety of complement and adhesion molecules, and form domain interactions with other proteins ([28]). Thus, the polymorphism in this domain is likely to affect important protein-protein interactions responsible for SELP-associated signal transduction processes.

Seven additional SNPs fell into the second category. Among this group of SNPs, it is noteworthy that 2 additional SNPs were found to cause amino acid changes in their respective proteins: the W58R polymorphism in KLRG1 (killer cell lectin-like receptor subfamily G, member 1 [KLRG-1] gene) and the S103C polymorphism in KIR2DS4 (killer cell Ig-like receptor, 2 domains, short cytoplasmic tail 4 gene). Moreover, the C to G polymorphism in the promoter of TNFSF4 (gene for tumor necrosis factor superfamily 4, encoding the OX40 ligand) is predicted to alter the binding site for the c-MycfMax transcription factor, as indicated in the TRANSFAC database ([25]). The Bayesian design of the microarray and rigorous multitest correction analysis in the present study assured that with relatively modest numbers of samples, the design of the study resulted in high-confidence findings.

FPRP. Although there are a variety of study and analysis designs currently used in linkage and association studies, only a few involve rigorous statistical methods ([15][16]). It is also quite common that replication studies or even reanalysis of the published data do not confirm the original conclusions. We therefore thought it important to analyze our present results using a different statistical approach. Because of the Bayesian methodology applied in this study at the outset (during the gene selection for the chip design), we used FPRP, a recently described method of Bayesian data analysis ([24]), for comparison with the FDR q values analysis. We established 4 categories for ranking the SNPs and estimating Bayesian prior probability values.

In the first category (pathway score), the ranking was done according to the number of times the gene was picked up by the gene searching programs from the databases (51). This ranking ranged from 0 to 9, with 9 being the maximum-scoring SNP; SNPs scored 0 were included because of chromosome locations (51). For example, rs3917815 (SELP) was picked up in 4 different key word searches, giving it a pathway score of 4 and a normalized pathway score of 0.44 (51). In the second category (gene location score), the ranking was done on the basis of established linkage with SLE ([2-11]). This ranking ranged from 0 to 5. High scores were assigned to genes based on the distance from the center of a linkage peak confirmed in at least 2 studies (e.g., SELP); genes that were further away received lower scores, and genes that were outside established linkage peaks received a score of 0. However, specific genes that were outside linkage peaks but confirmed to be involved in the genetic predisposition to SLE (e.g., PTPN22) received high scores. Next, each of the 1,024 genes was ranked by the investigators, using the description and Gene Ontology function, with respect to its likelihood of being associated with SLE based on available evidence in the literature (gene function score, range 0-10). Last, each SNP was ranked (SNP rank, range 0-5) according to its correspondence to a functionally identifiable region, (e.g., coding exon, promoter).

Because no distinct relative importance could be assigned a priori to the 4 categories, each score was normalized to a range of 0-1; all scores were then summed to yield the total score (51). Thus, theoretically, the maximum possible value for the total score is 4; in practice, the highest scoring SNP had a total score of 3.16 (TNFSF4 [rs1234314]). Next, all of the SNPs were ranked from 1 to 9,412 based on their total score and divided into 4 groups for assignment of Bayesian prior probabilities: top 1% (94 SNPs), top 5%, top 25%, and the rest, following a published algorithm ([24]). Wacholder et al ([24]) have suggested that when considering one or a few candidate polymorphisms, 0.1 should be viewed as the highest value of prior probability that any given polymorphism is true, and 0.01 as a modest value. In order to take into account multiple testing, we adopted a more conservative approach, in which prior estimates corresponded to a likelihood that 2 SNPs from each group would be described by alternative hypotheses. Thus, for the top 94 SNPs (top 1%) we assigned the prior probability of 0.02, for the top 5% the prior probability of 0.005, for the remaining top 25% the prior probability of 0.001, and for the rest the prior probability of 0.0003.

The powers for the TDTs were calculated according to method 4 described by Iles ([29]), using the frequency of the genotypes among affected children versus the expected frequency given Mendelian segregation to estimate genotype relative risk when possible, and conservatively assuming a genotype relative risk of 2:1:1 when it was not possible to estimate. Allele frequencies were estimated using the observed frequency of the major and minor SNP allele in the parents (51).

Wacholder et al ([24]) recommended designating genes with an FPRP of <0.5 as noteworthy. The 2 top genes (SELP and IRAK1) were the same in both analyses and had an FPRP or a q value of <0.05 in both cases. The FPRP procedure indicated as noteworthy 3 additional genes not identified in the q values analysis: BAT2 (gene for HLA-B-associated transcript 2), BF (gene for B-factor, properdin), and PTPN22; however, because we decided a priori to use q values to determine noteworthy genes, we do not argue for their significance here. It is also of interest that the average scores for SNP rank, gene location, and gene function for the genes were relatively high and similar (0.63, 0.62, and 0.52, respectively), emphasizing that each of these factors contributed significantly to the gene selection process, whereas the average pathway score (0.23) was considerably lower, reflecting the more general automated approach in the original gene selection.

It has been suggested that Bayesian analysis can be viewed in terms of the data from a study moving the field from the initial amount of information (Bayesian prior probabilities) to an increase in knowledge as reflected in the posterior probabilities ([30]), which in the present study were paralleled by FPRP values. Thus, in the case of IRAK1, for example, the prior probability that the null hypothesis (noninvolvement of IRAK1 in SLE) is true was 99.9%. This prior judgment was modified to the posterior probability of IRAK1 involvement in SLE being at least 98.47% (comparison of Bayesian prior probability and FPRP) (51). Finally, regarding the problem of multiple hypothesis testing in association studies, Colhoun et al suggested that in the presence of prior evidence of association, P values of 5×10⁻⁴ or smaller can be considered significant ([15]). Applying this simple criterion to our TDT results yielded exactly the same 9 genes.

### Discussion

Childhood-onset SLE presents a unique subgroup of patients for genetic study, because earlier disease onset, a more severe disease course, a greater frequency of family history of SLE, and a lesser effect of sex hormones in disease development ([31][3]) may imply an increased likelihood of expressing the genetic etiology. Most previous genetic studies were performed in patients with adult-onset disease. To our knowledge, this is the first study to use childhood-onset SLE cases and their parents.

We present herein a novel strategy using a combination of state-of-the-art hardware and analysis methods to investigate the genetics of a complex disease. The investigation is initiated by a bioinformatics-driven design of a custom-made chip that incorporates close to 10,000 SNPs derived from 1,000 selected genes. A variety of statistical data analysis methods have been used in studies reported in the current literature, with an all-too-common inability to replicate results of a different study or even a similar study using a different analysis method. In the present investigation, we used 2 fundamentally different methods for data analysis and obtained similar results. Overall, the study identified 2 new genes that were highly significantly associated with SLE, as well as 7 additional genes as candidates for followup investigation. The design of the microarray and rigorous multitest correction analysis assured that with a relatively modest number of samples, the study would yield high-confidence findings.

The most significant associations found in the present study were with polymorphisms at Asn/Ser amino acid 673 in SELP and Cys/Ser amino acid 203 in IRAK1. Seven additional SNPs demonstrated association, although not to a great enough level that they can be considered as proven. These SNPs and the respective genes in which they are found are prime candidates for further confirmation studies.

Although genetic association between SELP or IRAK1 and SLE has not been reported previously, both are attractive candidates. Indeed, P-selectin, a transmembrane protein expressed on activated platelets and endothelial cells, is an adhesion receptor for neutrophils, monocytes, and T lymphocytes ([33]). The interaction between P-selectin on endothelial cells and its ligands on T lymphocytes is responsible for the migration of these cells into inflamed tissue ([33]). Levels of platelet-leukocyte complexes as well as soluble P-selectin have been found to be significantly elevated in SLE patients ([34]). Since kidney involvement is one of the most devastating complications of SLE, it is notable that expression of both glomerular and interstitial P-selectin was up-regulated in various forms of proliferative glomerulonephritis including lupus nephritis ([35]). A recent study by He et al ([36]) showed that P-selectin-deficient MRL/lpr mice had accelerated development of glomerulonephritis and early mortality, and expression of monocyte chemotactic protein 1 (MCP-1) was increased in the kidneys and in supernatants of lipopolysaccharide-stimulated renal endothelial cells from these mice. These observations raise the possibility that expression of P-selectin is important for modulating the progression of glomerulonephritis, perhaps by down-regulating endothelial MCP-1 expression.

IRAK-1 is a serine/threonine protein kinase involved in the signaling cascade of the Toll/interleukin-1 receptor (TIR) family ([37]). The TIR family comprises the interleukin-1 (IL-1) receptor subfamily, recognizing the endogenous proinflammatory cytokines IL-1 and IL-18, and the members of the Toll-like receptor subfamily, recognizing pathogen-associated molecular patterns. A hallmark of the TIR family is the cytoplasmic TIR domain, which serves as a scaffold for a series of protein-protein interactions that result in the activation of a unique and exclusive signaling module consisting of myeloid differentiation factor 88, IRAK family members, and Toll-interacting protein. Subsequently, several central signaling pathways of the innate and adaptive immune system are activated in parallel, the activation of NF-B being the most prominent event of the inflammatory response ([37]). IRAK1 is considered to serve as the on-switch of the signaling complex by linking the receptor complex to the central adapter/activator protein tumor necrosis factor receptor-associated factor 6, and also as the off-switch of the complex by its autoinduced removal from the complex ([38]).

The C203S (Cys Ser) polymorphism in the IRAK1 gene is not a part of any currently known functional domain of the protein. However, the rather dramatic changes in the physicochemical properties of the amino acid substitution may suggest an associated functional change. The extensive involvement of IRAK1 in regulation of the immune response makes its association with SLE potentially important and a prime candidate for followup genetic and functional studies.

The W58R polymorphism in KLRG1 and the S103C polymorphism in KIR2DS4 suggest involvement of natural killer (NK) cells in the genetic predisposition to SLE. Both KLRG-1 and KIR2DS4 are expressed on NK cells and subsets of activated T lymphocytes. KIR2DS4 is an activating NK receptor molecule that enhances lysis by NK cells expressing KIR2DS4 ([39]), while KLRG-1-expressing NK cells show decreased proliferative activity ([40]). SLE patients, including those with childhood-onset disease, exhibit quantitative and qualitative alterations in NK cells ([41][42]). The genetic association of SLE with KLRG1 and KIR2DS4 in the present study, together with previous findings that first-degree relatives of SLE patients ([43]) and healthy monozygotic cotwins of SLE patients ([44]) show reduced numbers and activity of NK cells, suggests that this phenotype might be involved in disease causation rather than being a consequence of the disease process.

Neutrophilic cytosolic factor 2 (NCF-2) is an essential component of the NADPH oxidase enzyme complex in phagocytic leukocytes. Its importance in host innate immunity is demonstrated by the finding of recurrent infections in individuals with chronic granulomatous disease resulting from genetic defects in components of the NADPH complex, including NCF2 (the gene for NCF-2) ([45]). However, phagocyte-generated reactive oxidants can also contribute to host injury associated with inflammation. Furthermore, the association between SLE and NCF2 gene suggested from the present results may be related to the overexpression pattern of various neutrophil genes observed in gene expression profiles of patients with childhood-onset SLE ([46]).

Although several members of the tumor necrosis factor and tumor necrosis factor receptor families have been implicated in the pathogenesis of SLE (including the TNFRSF6 gene suggested in the present study), the data presented herein provide the first direct evidence of genetic association between SLE and TNFSF4, encoding the OX40 ligand. Interaction between OX40 and its ligand is involved in costimulation of T and B lymphocyte activation and in T cell adhesion to endothelium. Immunohistologic study of renal biopsy specimens from patients with lupus nephritis demonstrated an abundant presence of OX40 ligand in all cases of proliferative lupus nephritis, in a unique granular distribution and colocalized with subepithelial immune deposits ([47]).

It is also noteworthy that 3 of the 5 highest-scoring genes in the present study are closely colocalized (1q24.2-1q25.3, within a stretch of 14 Mb) (51), suggesting a strong association of this region with SLE and making it a prime candidate for followup fine mapping studies. The linkage of SLE with this chromosomal region has been reported previously ([4][9][45]).

Our results also corroborate the previously reported association between SLE and IRF5 (gene for interferon regulatory factor 5) ([48][49]) and emphasize the importance of the interferon-pathway in SLE ([50]). Finally, the association of SLE with PTPRT (gene for protein tyrosine phosphatase receptor type T) is a novel addition to the known connection between SLE and PTPN22 ([12]) and underscores the importance of lymphocyte tyrosine phosphatase regulation.

The present results demonstrate the powerful potential of this novel combination of up-to-date biotechnology and bioinformatics methods in the search for genetic origins of common complex diseases. Furthermore, the discovery of new SLE-associated genes opens promising new directions for understanding the genetic foundations of and ultimately treating this relatively common and devastating disease.

### REFERENCES

1. Russ V, Hochberg M C. The epidemiology of lupus erythematosus. In: Wallace D J, Hahn B H, editors. Dubois' lupus erythematosus. 6th ed. Philadelphia: Lippincott Williams & Wilkins; 2002. p. 65-83.
2. Gaffney P M, et al. A genome-wide search for susceptibility genes in human systemic lupus erythematosus sib-pair families. Proc Natl Acad Sci USA 1998; 95: 14875-9.
3. Moser K L, et al. Genome scan of human systemic lupus erythematosus: evidence for linkage on chromosome 1q in African-American pedigrees. Proc Natl Acad Sci USA 1998; 95: 14869-74.
4. Shai R, et al. Genome-wide screen for systemic lupus erythematosus susceptibility genes in multiplex families. Hum Mol Genet 1999; 8: 639-44.
5. Gaffney P M, et al. Genome screening in human systemic lupus erythematosus: results from a second Minnesota cohort and combined analyses of 187 sib-pair families. Am J Hum Genet 2000; 66: 547-56.
6. Gray-McGuire C, et al. Genome scan of human systemic lupus erythematosus by regression modeling: evidence of linkage and epistasis at 4p16-15.2. Am J Hum Genet 2000; 67: 1460-9.
7. Tsao B P. Update on human systemic lupus erythematosus genetics. Curr Opin Rheumatol 2004; 16: 513-21.
8. Graham R R, et al. Genetic linkage and transmission disequilibrium of marker haplotypes at chromosome 1q41 in human systemic lupus erythematosus. Arthritis Res 2001; 3: 299-305.
9. Cantor R M, et al. Systemic lupus erythematosus genome scan: support for linkage at 1q23, 2q33, 16q12-13, and 17q21-23 and novel evidence at 3p24, 10q23-24, 13q32, and 18q22-23. Arthritis Rheum 2004; 50: 3203-10.
10. Nath S K, et al. Linkage at 12q24 with systemic lupus erythematosus (SLE) is established and confirmed in Hispanic and European American families. Am J Hum Genet 2004; 74: 73-82.
11. Nath S K, et al. Systemic lupus erythematosus (SLE) and chromosome 16: confirmation of linkage to 16q12-13 and evidence for genetic heterogeneity. Eur J Hum Genet 2004; 12: 668-72.
12. Kyogoku C, et al. Genetic association of the R620W polymorphism of protein tyrosine phosphatase PTPN22 with human SLE. Am J Hum Genet 2004; 5: 504-7.
13. Risch N, et al. The future of genetic studies of complex human diseases. Science 1996; 273: 1516-7.
14. Botstein D, Risch N. Discovering genotypes underlying human phenotypes: past successes for mendelian disease, future approaches for complex disease. Nat Genet 2003; 33 Suppl: 228-37.
15. Colhoun H M, et al. Problems of reporting genetic associations with complex outcomes. Lancet 2003; 361: 865-72.
16. Freimer N, et al. The use of pedigree, sib-pair and association studies of common diseases for genetic mapping and epidemiology. Nat Genet 2004; 36: 1045-51.
17. Tan E M, et al. The 1982 revised criteria for the classification of systemic lupus erythematosus. Arthritis Rheum 1982; 25: 1271-7.
18. Hochberg M C, for the Diagnostic and Therapeutic Criteria Committee of the American College of Rheumatology. Updating the American College of Rheumatology revised criteria for the classification of systemic lupus erythematosus [letter]. Arthritis Rheum 1997; 40: 1725.
19. Gladman D, et al. The development and initial validation of the Systemic Lupus International Collaborating Clinics/American College of Rheumatology Damage Index for systemic lupus erythematosus. Arthritis Rheum 1996; 39: 363-9.
20. Hardenbol P, et al. Multiplexed genotyping with sequence-tagged molecular inversion probes. Nat Biotechnol 2003; 21: 673-8.
21. Hardenbol P, et al. Highly multiplexed molecular inversion probe genotyping: over 10,000 targeted SNPs genotyped in a single tube assay. Genome Res 2005; 15: 269-75.
22. Spielman R S, et al. Transmission test for linkage disequilibrium: the insulin gene region and insulin-dependent diabetes mellitus (IDDM). Am J Hum Genet 1993; 52: 506-16.
23. Storey J D, et al. Statistical significance for genomewide studies. Proc Natl Acad Sci USA 2003; 100: 9440-5.
24. Wacholder S, et al. Assessing the probability that a positive report is false: an approach for molecular epidemiology studies. J. Natl Cancer Inst 2004; 96: 434-42.
25. Kel A E, et al. MATCH: a tool for searching transcription factor binding sites in DNA sequences. Nucleic Acids Res 2003; 31: 3576-9.
26. Cordell H J, et al. Genetic association studies. Lancet 2005; 366: 1121-31.
27. Benjamini Y, et al. Controlling the false discovery rate: a practical and powerful approach to multiple testing. J R Stat Soc 1995; 85: 289-300.
28. Kirkitadze M D, et al. Structure and flexibility of the multiple domain proteins that regulate complement activation. Immunol Rev 2001; 180: 146-61.
29. Iles M M. On calculating the power of a TDT study—comparison of methods. Ann Hum Genet 2002; 66: 323-8.
30. Goodman S N. Of P-values and Bayes: a modest proposal. Epidemiology 2001; 12: 295-7.
31. Cassidy J T. Childhood onset SLE, In Cassidy J T, Petty R E, editors. Textbook of pediatric rheumatology. Philadelphia: Elsevier Saunders; 1996. p. 329-406.
32. Lehman T J. SLE in childhood and adolescence. In: Wallace D J, Hahn B H, editors. Dubois' lupus erythematosus. 6th ed. Philadelphia: Lippincott Williams & Wilkins; 2002. p. 863-84.
33. Ley K. The role of selectins in inflammation and disease. Trends Mol Med 2003; 9: 263-8.
34. Joseph J E, et al. Increased circulating platelet-leucocyte complexes and platelet activation in patients with antiphospholipid syndrome, systemic lupus erythematosus and rheumatoid arthritis. Br J Haematol 2001; 115: 451-9.
35. Segawa C, et al. In situ expression and soluble form of P-selectin in human glomerulonephritis. Kidney Int 1997; 52: 1054-63.
36. He X, et al. Deficiency of P-selectin or P-selectin glycoprotein ligand-1 leads to accelerated development of glomerulonephritis and increased expression of CC chemokine ligand 2 in lupus-prone mice. J Immunol 2006; 177: 8748-56.
37. Martin M U, et al. Summary and comparison of the signaling mechanisms of the Toll/interleukin-1 receptor family. Biochim Biophys Acta 2002; 1592: 265-80.
38. Kollewe C, et al. Sequential autophosphorylation steps in the interleukin-1 receptor-associated kinase-1 regulate its availability as an adapter in interleukin-1 signaling. J Biol Chem 2004; 279: 5227-36.
39. Katz G, et al. MHC class I-independent recognition of NK-activating receptor KIR2DS4. J Immunol 2004; 173: 1819-25.
40. Voehringer D, et al. Lack of proliferative capacity of human effector and memory T cells expressing killer cell lectinlike receptor G1 (KLRG1). Blood 2002; 100: 3698-702.
41. Erkeller-Yusel F, et al. Lymphocyte subsets in a large cohort of patients with systemic lupus erythematosus. Lupus 1993; 2: 227-31.
42. Yabuhara A, et al. A killing defect of natural killer cells as an underlying immunologic abnormality in childhood systemic lupus erythematosus. J Rheumatol 1996; 23: 171-7.
43. Green M R, K et al. Natural killer cell activity in families of patients with systemic lupus erythematosus: demonstration of a killing defect in patients. Clin Exp Immunol 2005; 141: 165-73.
44. Stohl W, et al. Impaired recovery and cytolytic function of CD56+ T and non-T cells in systemic lupus erythematosus following in vitro polyclonal T cell stimulation: studies in unselected patients and monozygotic disease-discordant twins. Arthritis Rheum 1996; 39: 1840-51.
45. Francke U, et al. Genes for two autosomal recessive forms of chronic granulomatous disease assigned to 1q25 (NCF2) and 7q11.23 (NCF1). Am J Hum Genet 1990; 47: 483-92.
46. Bennett L, et al. Interferon and granulopoiesis signatures in systemic lupus erythematosus blood. J Exp Med 2003; 197: 711-23.
47. Aten J, et al. Strong and selective glomerular localization of CD134 ligand and TNF receptor-1 in proliferative lupus nephritis. J Am Soc Nephrol 2000; 11: 1426-38.
48. Sigurdsson S, et al. Polymorphisms in the tyrosine kinase 2 and interferon regulatory factor 5 genes are associated with systemic lupus erythematosus. Am J Hum Genet 2005; 76: 528-37.
49. Graham R R, et al. A common haplotype of interferon regulatory factor 5 (IRF5) regulates splicing and expression and is associated with increased risk of systemic lupus erythematosus. Nat Genet 2006; 38: 550-5.
50. Baechler E C, et al. The emerging role of interferon in human systemic lupus erythematosus. Curr Opin Immunol 2004; 16: 801-7.
51. Jacob, C, et al., Identification of Novel Susceptibility Genes in Childhood-Onset Systemic Lupus Erythematous Using a Uniquely Designed Candidate Gene Pathway Platform. Arthritis and Rheumatism, 2007; 56(12):4164-4173.

### Example 3

Many common disorders have genetic components which convey increased susceptibility. While linkage and association analyses have been quite successful in identifying rare variants with high penetrance, such as in Huntington's disease [1] and some forms of cancer [2], the ability of these approaches to detect common variants with more modest effects (less penetrance) is limited. Frequent alleles with modest genetic effects play important roles in the susceptibility to most diseases. For example, most autoimmune disorders involve multiple alleles of different genes with individual low penetrance which also interact with environmental factors to produce the final disease phenotype. Dissecting the disease phenotype into the individual genes and associated alleles that are responsible is crucial for understanding the mechanism of the disease and ultimately developing treatment modalities [3]. To this end, many researchers have been using genome-wide approaches to locate Single Nucleotide Polymorphisms (SNPs) that are in disequilibrium with a disease-causing allele, or associated with a disease phenotype [3]. Unfortunately, the requirement to completely scan the entire genome with sufficient SNP coverage required to achieve an appropriate study-wide power as well as the concomitant increase in number of subjects required to overcome the multiple-testing effect makes this type of study prohibitively expensive. Indeed, many association studies are under-powered, which leads to low reproducibility, compounded additionally by publication bias [4-7].

There are two general methods to reduce the number of SNPs tested in a search for variants which convey susceptibility. The first is to reduce the number of SNPs necessary to cover the entire genome by maximizing the information conveyed by each individual SNP. This process involves the elimination of redundant SNPs whose state is already determined with high probability by other SNPs in the study [8,9]. The second method is to use prior available information to select genes and genomic regions likely to be involved in a disease and testing the most likely genes and regions in preference to the least likely. While this approach does have advantages over a whole-genome study, specifically in financial cost, time required and a smaller number of subjects needed to assure reasonable power, it does have disadvantages in that it does not select candidate genes which are not associated with biological functions or genomic regions currently thought to be related or linked to a disease.

In order to determine which genes or genomic regions are likely to be associated with the disease, it is necessary to connect genes and genomic areas with available literature on the disease and disease-associated pathways. The implementation we present here relies on experts to build a list of keywords and genomic areas from the available literature and expert knowledge coupled with publicly available databases to connect keywords and genomic areas to specific genes. An alternative using automated information extraction techniques which do not rely on expert knowledge is presented in the discussion. This experimental design, where the genes (and consequently, SNPs within those genes) are selected using prior available information allows for the determination of the prior probability that a particular gene is associated with a disease.

Once specific genes have been selected, the SNPs used in the study need to be selected. A popular class of methods is the tagSNP methods, which attempt to reduce the number of SNPs while maximizing the information that each SNP represents by grouping SNPs that are in linkage disequilibrium with each other and in the same phase (see [8,9] for a comparison of different methods). The genes suggested by our program and its associated method do not necessarily require such powerful techniques, but discarding redundant SNPs will be useful in maximizing power while reducing cost. Beyond discarding non-informative SNPs, special importance should be placed on functional SNPs as they are more likely to actually represent the disease allele in question.

The method presented here uses a combination of automated and manual approaches to maximize the power of a study using SNPs. The method has the following steps:

1. Use expert knowledge and literature to identify a set of keywords (biological functions and genetic regions) which have high prior probability of being associated with the disease.
2. Use publicly available databases to select genes that reference the set of keywords.
3. Rank the identified genes based on their prior probability of association using the selection results and expert knowledge.
4. Choose an appropriate number of genes for SNP selection and association studies based on the number of cases available, monetary, and time constraints of the study while maintaining acceptable study-wide power.
5. Conduct study.
6. Analyze results, optionally using the prior probability of association obtained during genetic selection.

Implementation. The application is separated into a series of separate scripts, each of which has a specific function, and operate in a specific order 15).

Once a set of keywords (biological functions and genomic locations judged to be associated or otherwise involved with the disease) has been identified by expert knowledge of the disease in question, publicly available databases (currently NCBI, GeneCards, and Harvester are defaults, but Uniprot and Ensembl are also supported) are queried in series with delays between each query as appropriate for each database to avoid overloading them. Because there is no well documented common interchange format for these databases, the get_series of scripts download the HTML (or XML) which the search requests generate and save it. To avoid putting unreasonable demands on the databases, the get_scripts limits the number of requests they make per minute.

After all of the HTML (or XML) has been retrieved, the next series of scripts (parse_) are run which use the HTML::Parse (or XML::Parser::Expat) module to extract the gene name, accession number for the reference sequence, genomic location, alias(es), function(s), and description.

These files are then combined into a single file by combine_results that tracks what database and keyword resulted in which genes. The aliases from all databases are joined. Gene descriptions are retrieved from each database; the longest description is retained in the final list. For example, Harvester, when queried with “adhesion”, returns VCAM1 (amongst others). Harvester also returns VCAM1 when queried with “inflammation”. GeneCards also returns VCAM1 for these two queries. combine_results would then indicate that GeneCards returned VCAM1 twice, Harvester returned VCAM1 twice, and it was returned for “adhesion” and “inflammation”. At the same time three separate weighting procedures are applied to order the genes. First, a simple approach, dubbed “rzscore”, simply counts the number of times that a gene was returned by unique search terms, and orders the results from most number of term matches to least. The second, allows user-specified weights to be applied to each keyword, and orders the results by the sum of the weights of the corresponding keywords which returned a result. The third method automatically weights the keywords to avoid over-counting keywords which are entirely subsets of other keywords. It does this by dividing the number of results returned by a keyword by the sum of the number of results returned by that keyword and any other keyword, including itself.

To facilitate easier use of this collection of meta-search utilities, a script which binds them all together has also been provided, called function2gene, which takes a set of keywords, an optional set of databases to query, and returns the complete tabulated results to the specified directory.

By using perl and existing modules (WWW::Mechanize, HTML::TreeBuilder and XML::Parser) the actual number of lines (and resulting implementational complexity) of the codebase is greatly reduced, as custom code to deal with form submission as well as HTML and XML parsing did not need to be written. This approach also allows the scripts to be slightly less dependent on the exact output format of the sites which are searched. Splitting the retrieval and parse stages also allows for extracting additional information from the search results by modifying the parser and reparsing the results without waiting to retrieve results from the remote databases again.

### Results and Discussion

The system described above was utilized to generate a list of genes which were then used to select SNPs in a study of childhood-onset Systemic Lupus Erythematosus (SLE). SLE is a debilitating multi-system autoimmune disorder affecting ≈0.1% of the North American population. An initial search using a set of 31 keywords (consisting of biological functions and chromosomal regions) selected by expert knowledge returned 6798 genes with various contributions from the three databases used (15). It is important to note that the results obtained are temporally-sensitive; as databases are updated different sets of genes will be returned. In every case a single database did not retrieve all the genes found by other databases, demonstrating the need to query multiple databases. The substantial contribution made by each database in identifying the candidate genes demonstrates that each of the databases is required to maximize the number of candidate genes discovered, though there are likely results which are still not captured by the set of databases queried. As new databases come into prominence, Function2Gene can be extended to query them as well. The top 1204 genes (of which 836 were returned by GeneCards, 699 by Harvester, and 135 by NCBI) were used to select 9412 SNPs. The number of genes to select was dictated by the capacity of the chip (≈10,000 SNPs), and a decision to have approximately ten SNPs per gene on average. The choice of SNPs to genotype within the selected genes was based on available information from databases including the Human Haplotype Mapping Project (HapMap) with priority given to SNPs with high heterozygosity in two or more relevant ethnicities and to SNPs representing amino acid coding variants. The selected SNPs were then cross-checked against the accumulated SNP validation test results available at our industrial collaborator (ParAllele Biosciences), an active participant of the International HapMap project.

Using the selected SNPs, 251 nuclear families consisting of both parents and the affected child (full trios) were genotyped. The analysis of the genotypes of the 251 trios using Transmission Disequilibrium Test (TDT) followed by False Discovery Rate (FDR) multi-test correction yielded 9 noteworthy genes, that are associated with SLE with FDR less than 0.5; two of these genes were highly significant, with FDR less than 0.05 [10].

Using Bayesian methodologies, the impact of pre-existing knowledge of a disease on the discovery of genes associated with the disease can be increased, as the posterior probability of association with the disease can be modified in accordance with its prior probability as reported by function2gene. The False Positive Report Probability (FPRP) measure is one such method which uses the prior probability of association, which can be calculated from the results of the keyword-based gene selection, to modify the posterior probability of association. Using Bayes' theorem

$$( P ( A \vert B ) = \frac{P ( B \vert A ) P ( A )}{P ( B )} )$$

FPRP determines the probability of the null hypothesis (no association) being true given a test statistic greater than Zα (that is to say p≦α), knowing power (1-β), the prior probability of association (Π), and the probability of the measured data given that the null hypothesis is true (p) [11]:

$$FPRP = P ( {H}_{0} is true \vert T > {Z}_{a} ) = \frac{p ( 1 - π )}{p ( 1 - π ) + ( 1 - β ) π}$$

One method of calculating prior probability based on the keyword based gene selection is to order the SNPs according to number of times they were returned by different keywords, taking into account the biological relevance of the SNP, and then apply a continuous function such that the higher ranked SNPs have a greater prior probability of association than the lower ranked SNPs, and the sum of the probability of association is the prior estimate of the total number of SNPs in the search believed to be associated with the disease. An alternative method is to order the SNPs in the same manner, and then place them in to different groups, assigning the same prior probabilities to each SNP in a group while controlling the sum of the prior probabilities assigned. For example, assuming 10,000 SNPs, 10 of which are believed to be associated, assign priors of Π=0.025 for the top 1%, 6.25×10−3 to the next top 4%, 1.25×10−3 to the next top 20%, and 3.33×10−4 to the remaining 75%. In this manner the multiple testing effect is controlled while maximizing the effect of the prior available information. Applying FPRP [11] to the results of the TDT test with a prior assumption of 8 associated SNPs yielded 12 noteworthy genes, including all 9 obtained with the FDR corrections, and the same two significant genes [10]. An existing web-based program which is functionally similar to the methodology presented here is the Disease Candidate Gene search of SNPs3d. Using the keywords chosen by SNPs3d for three diseases, diabetes, pancreatic cancer, and Alzheimer disease, we have compared the results obtained by SNPs3d and Function2Gene in Table 2. The majority of high ranking genes returned by SNPs3d are also returned by Function2Gene, but Function2Gene returns a far greater number of genes.

Future advancements of the approach presented here could be made by the use of more powerful literature mining techniques which would reduce (or even eliminate) the need for expert information on the nature, pathology, and biology of the disease to generate a list of keywords and discard spurious results. Such approaches would also reduce the reliance of this approach on the contents of stewarded fields in the databases, enabling novel associations as well as incorrect associations to be discerned. For example, Named Entity Recognition (NER) and Relationship Extraction (RE) could be used in tandem to elucidate connections between diseases and genes directly. NER identifies biologically-relevant entities (like genes and proteins) from literature using techniques such as hidden Markov models and dictionaries. Once entities have been identified, RE can identify the relationship and/or connection between entities using the proximity of entities (and the re-occurrence of entities in close proximity), along with rule base systems and full predicate/subject grammars [12-14]. It would then be possible to walk the relationship tree, using the probabilities between each node of the tree connecting specific genes and a disease (with intervening genes, proteins, and biological pathways in between), and then ordering the resultant genes by the probability of their connection which should be directly proportional to the prior probability of association.

### Conclusion

The use of available prior information to decrease the size of the problem space for gene identification in complex polygenic disorders is an as yet underutilized technique. The methodology and the programs presented here use data mining techniques to retrieve from a few databases a number of genes with high prior probability of being associated with the disease. The program also allows to order genes by relevance, thereby allowing the problem space to be greatly reduced, increasing the power of the study and thus increasing the likelihood of successfully finding associated genes.

### REFERENCES

1. MacDonald M E, et al. A novel gene containing a trinucleotide repeat that is expanded and unstable on Huntington's disease chromosomes. The Huntington's Disease Collaborative Research Group. Cell 1993, 72(6):971-83.
2. Hall J M, et al. Linkage of early-onset familial breast cancer to chromosome 17q21. Science 1990, 250(4988):1684-9.
3. Risch N, et al. The future of genetic studies of complex human diseases. Science 1996, 273(5281):1516-7.
4. Colhoun H M, M et al. Problems of reporting genetic associations with complex outcomes. Lancet 2003, 361(9360):865-72.
5. Ioannidis J P, et al. Replication validity of genetic association studies. Nat Genet 2001, 29(3):306-9.
6. Ioannidis J P A: Why most published research findings are false. PLoS Med 2005, 2(8):e124.
7. Nannya Y, et al. Evaluation of genome-wide power of genetic association studies based on empirical data from the HapMap project. Hum Mol Genet 2007, 16(20):3494-505.
8. Burkett, G et al. A comparison of five methods for selecting tagging single-nucleotide polymorphisms. BMC Genetics 2005, 6(Suppl 1):S71.
9. Butler, B et al. Strategies for selecting subsets of single-nucleotide polymorphisms to genotype in association studies. BMC Genetics 2005, 6(Suppl 1):S72.
10. Jacob C O, et al. Identification of novel susceptibility genes in childhood-onset systemic lupus erythematosus using a uniquely designed candidate gene pathway platform. Arthritis Rheum 2007, 56(12):4164-73.
11. Wacholder S, et al. Assessing the probability that a positive report is false: an approach for molecular epidemiology studies. J Natl Cancer Inst 2004, 96(6):434-42.
12. Erhardt R A A, et al. Status of text-mining techniques applied to biomedical text. Drug Discov Today 2006, 11(7-8):315-25.
13. Ananiadou S, et al. Text mining and its potential applications in systems biology. Trends Biotechnol 2006, 24(12):571-9.
14. Spasic I, et al. Text mining and ontologies in biomedicine: making sense of raw text. Brief Bioinform 2005, 6(3):239-51.
15. Armstrong, D., et al. Function2Gene: A gene selection tool to increase the power of genetic association studies by utilizing public databases and expert knowledge. BMC Bioinformatics 2008, 9:311.

### Example 4

Systemic lupus erythematosus (SLE) is a debilitating multisystem autoimmune disorder affecting ≈0.1% of the North American population, mainly females, characterized by chronic inflammation and extensive immune dysregulation in multiple organ systems, associated with the production of autoantibodies to a multitude of self-antigens (1). The prevalence of SLE varies among ethnic populations (higher in non-Caucasians) and is likely attributable to ethnic differences in genetic susceptibility. Despite many advances in recent years, the pathogenesis of SLE remains largely unclear.

Genetic approaches have gained much power and popularity in identifying the component mechanism(s) underlying the pathogenesis of common human diseases. Forward genetic approaches, in which human populations are studied to identify the genes involved in disease processes, have inherent shortcomings for the analysis of common diseases involving multiple genes because each gene contributes modestly, often in interaction with environmental factors. On the other hand, reverse genetic approaches—in which a gene is characterized by perturbing it in an experimental system, and then elucidating its effect on the trait of interest—have their own significant limitations. Often, such experimental approaches take place in an oversimplified context where potential interactions between the gene of interest and the genetic background or the environment are eliminated and data interpretation may be confounded by the impact of the gene on cell and organismal development. In the present study, a combined forward and reverse genetic approach is pursued, resulting in the unequivocal identification of the gene IRAK1 as an important risk factor for SLE, with a critical role in disease pathogenesis.

We have recently developed a set of programs that implement a combination of automated and manual approaches to maximize the power of gene association studies by using prior information to select and prioritize genes, to reduce the number of SNPs tested resulting in higher power, and to increase the likelihood of uncovering reproducible associations (2). We have previously used this bioinformatics-driven design for a custom-made platform incorporating ≈10,000 SNPs derived from ≈1,000 selected genes to genotype a sample of 753 subjects composed of 251 childhood-onset SLE trios (SLE patient and both parents) (3). Family-based transmission disequilibrium test (TDT) and multitest correction analyses showed a significant association between the IRAK1 gene on chromosome Xq28 and childhood-onset SLE (3).

In the present study, we have used a case-control association approach to test the hypothesis that IRAK1 is a candidate gene predisposing to SLE. To this end, we have tested an independent childhood-onset cohort of 769 childhood onset SLE patients, 5,337 North American adult-onset SLE subjects, and 5,317 healthy controls, each group being composed of 4 ethnicities (40). Childhood-onset SLE constitutes a unique subgroup of patients for genetic analysis because the earlier disease onset, the more severe disease course, the greater frequency of family history of SLE, and a lesser contribution of sex hormones in disease development (4, 5) may all translate to a higher genetic load or a more penetrant expression of this genetic load, and this may facilitate gene discovery relative to studies of the adult-onset disease. Therefore, we analyzed childhood-onset and adult-onset groups of SLE patients separately. To account for any potential confounding substructure or admixture, we performed principal component analyses (PCA) (6), as detailed in Methods. Excluding the outliers, the analyses resulted in low inflation factors in all ethnicities except Hispanic Americans, with only the latter requiring additional principal component correction.

There is an association of IRAK1 SNPs in four racial groups of childhood- and adult-onset SLE (40). It is noteworthy that the majority of the significantly associated SNPs are within a relatively small interval of 3.3 kb between intron 10 and intron 13 of the IRAK1 gene. Most of these SNPs show significance in multiple ethnicities (40). The classical Bonferroni correction and similar procedures for controlling the family-wise error rate for multiple testing are both too strict and inappropriate in studies such as the present one because they assume that each test is independent, whereas in actuality a complex and unknown mutual dependence exists among SNPs on the same gene (3, 7). Therefore, for multiple test correction we calculated estimates of the false discovery rate (FDR) q values by using the Benjamini-Hochberg procedure (8) considering the total number of SNPs tested and the 4 different ethnic groups (40). Combined p values were calculated from the per-ethnicity p value by using the Fisher method. 5 SNPs out of the 13 tested within the IRAK1 gene showed significant association with SLE in multiple ethnic groups after correction for multiple testing. There are a number of highly significant SNPs with combined p values reaching 10-10, and attaining 10-9 in individual ethnicities, corresponding to FDRs of 10-9 and 10-7, respectively.

Three of the 5 associated SNPs (rs2239673, rs763737, and rs7061789) overlap in both the childhood- and adult-onset SLE patients, suggesting a similar involvement of IRAK1 in both adult- and childhood-onset SLE. The odds ratio (ORs) of all significantly associated SNPs are in the same direction (>1), implying that there was no residual population stratification. It is also noteworthy that ORs for the associated SNPs, with the exception of rs5945174, are >1.5, a value that compares well with published associations in SLE and other similar complex human disorders (9-14). The most significantly associated SNPs are in a linkage-disequilibrium block that extends from intron 10 to intron 13 of the IRAK1 gene. Haplotype analyses in different racial groups show that the GGGG haplotype (defined as “G” at rs2239673, “G” at rs763737, “G” at rs5945174, and “G” at rs7061789) is significantly associated with disease in 3 of the 4 racial groups in adult-onset SLE and in 3 ethnicities in childhood-onset SLE (40). The p values for association reach 10-5 in children and 10-6 in adults. On the other hand, the AAAA haplotype is clearly associated with protection from disease.

Currently no human biological system is available that would allow one to ascertain an in vivo connection between IRAK1 and its biological relevance in SLE. To test this, we turned to the laboratory mouse, as mice lacking IRAK1 function and mice prone to spontaneous lupus have both been described on the same C57BL/6 (B6) genetic background (15-17). Recent studies have succeeded in defining the genetic basis of lupus in the NZB×NZW derived NZM mouse models, and have uncovered Sle1 on chromosome 1 and Sle3 on chromosome 7 as 2 of the most critical elements for disease in these models (16-20). By introgressing these intervals onto the relatively normal C57BL/6 (B6) background, the immunological properties of these 2 key loci have been elucidated (16, 17). Whereas a critical gene within the Sle1 interval, Ly108, breaches central B cell tolerance, resulting in anti-chromatin autoreactivity and lymphocytic activation (19), the Sle3 gene(s) contributes to SLE by activating myeloid cells, including dendritic cells (DCs) (20). Importantly, the combined action of these 2 loci leads to full-blown lupus and lupus nephritis, which is indistinguishable from the disease noted in the traditionally studied (NZB×NZW)F1 and NZM mouse models (18).

Because Sle1 and Sle3 represent 2 key complementary loci for SLE development, we evaluated the role of IRAK1 in mediating the contributions of these 2 loci to SLE pathogenesis. B6.Sle1^{z} mice (that were homozygous for the Sle1z allele) were bred to B6.IRAK1^{-/Y} mice (15), to eventually derive B6.Sle1^{z}.IRAK^{-/Y} mice. Because Sle1^{z} leads to spontaneous anti-nuclear antibody formation on the B6 background, notably anti-histone/DNA antibodies, splenomegaly, and spontaneous B cell and T cell activation (16), these phenotypes were first examined. Compared to age- and sex-matched B6.Sle1^{z} control, B6.Sle1^{z}.IRAK1^{-/Y} mice exhibited significantly reduced IgM and IgG autoantibodies to ssDNA, histone/DNA, and dsDNA (40). Likewise, B6.Sle1^{z}.IRAK1^{-/Y} mice also exhibited reduced spleen weights, total splenocyte counts, as well as total B cell and CD4-positive T cell counts, compared with the controls with an intact IRAK gene (40). In addition, the absence of IRAK1 also dampened the number of B cell blasts (as gauged by forward scatter analysis) (40) and reduced the numbers of activated CD4 T cells as assessed by surface CD69 expression (40). No differences were, however, noted in the expression of surface CD86 or CD69 on B cells from both strains. Collectively, the above findings indicate, that the absence of IRAK1 significantly attenuated the serological and cellular phenotypes attributed to the lupus susceptibility locus, Sle1.

Next, we proceeded to examine the impact of IRAK1 in mediating the lupus contributions of the second locus, Sle3. In the B6 background, Sle3^{z} leads to low-grade anti-nuclear serological autoreactivity, myeloid cell hyperactivity resulting in secondary activation of lymphocytes, and a modest degree of nephritis (17, 20). Compared to B6.Sle3^{z} controls, B6.Sle3^{z}.IRAK1^{-/Y} mice exhibited significantly reduced IgM and IgG anti-ssDNA and anti-dsDNA Abs (40), as well as milder or negligible renal disease, as evidenced by the reduced proteinuria and renal glomerular pathology (40). Moreover, these mice had reduced splenocyte numbers, including total T cells and B cells (40). A cardinal feature associated with Sle3^{z}, namely increased CD4:CD8 ratios, were normalized by the absence of IRAK1 (40). Because the above phenotypes had previously been attributed to the intrinsic impact of Sle3z on myeloid cells (20), these were examined next. Although the strains did not differ in absolute numbers of splenic myeloid cell subpopulations, interesting differences in their activation and maturation status were observed. In the absence of IRAK1, Sle3^{z} myeloid DCs and macrophages examined ex vivo from spleens showed reduced surface expression of CD80, but not CD40 or CD86 (40). These differences became more pronounced when bone marrow (BM)-derived DCs were examined. Thus Sle3^{z} BM-DCs deficient in IRAK1 exhibited reduced levels of several activation/maturation markers both basally and after TLR ligation using poly(I C) or CpG (40). The IRAK1-deficient B6.Sle3^{z} DCs also produced reduced levels of proinflammatory cytokines, such as TNF-α (40). Hence, all of the phenotypes previously attributed to the Sle3^{z} lupus susceptibility locus appear to be, at least partly, dependent upon IRAK1 function.

### Discussion

IRAK1 (interleukin-1 receptor associated kinase 1) is a serine/threonine protein kinase involved in the signaling cascade of the Toll/IL-1 receptor (TIR) family (21). The TIR family comprises the IL-1 receptor subfamily, recognizing the endogenous proinflammatory cytokines IL-1 and IL-18, and members of the Toll-like receptor (TLR) subfamily, which recognize pathogen-associated molecular patterns. A hallmark of the TIR family is the cytoplasmic TIR domain, which serves as a scaffold for a series of protein-protein interactions, which result in the activation of a unique and exclusive signaling module consisting of MyD88, IRAK family members, and Tollip. Subsequently, several central signaling pathways of the innate and adaptive immune systems are activated in parallel, the activation of NFκB being the most prominent event of the inflammatory response (21). Particularly noteworthy is the observation that IRAK1 is considered to be the “on-switch” of the signaling complex by linking the receptor complex to the central adapter/activator protein TRAF6, and also the “off-switch” of the complex because of its autoinduced removal from the complex (2). The extensive involvement of IRAK1 in the regulation of the immune response renders its association with SLE a prime candidate for careful genetic and functional analysis.

We envision the potential involvement of IRAK1 in at least the following 3 immune cell functions that have been reported to be aberrant in SLE. First, IRAK1 is involved in the induction of IFN-α and IFN-γ: the production of both types of IFN has been shown to be aberrant in SLE (23, 24). Second, IRAK1 is a pivotal regulator of the NFκB pathway. Abnormal NFκB activity in T lymphocytes from patients with SLE has been amply documented (25). Finally, a growing number of studies demonstrate a role for TLR activation in the pathogenesis of SLE, including the activation of anti-nuclear B cells and the subsequent immune complex formation (26). The murine studies presented in this communication resonate well with the earlier published literature on IRAK1.

The most significantly associated SNPs are in a linkage-disequilibrium block that extends from intron 10 to intron 13 of the IRAK1 gene, encompassing exons 11-13, which correspond to the C1 domain of IRAK1 (27). It has been shown that this domain is at least partially responsible for the interaction with signal transduction factors such as TRAF6 (28). Furthermore, a naturally occurring splice variant of IRAK1, IRAK1c, lacks exon 11 and most of exon 12 (27). A previous report suggests that IRAK1c may suppress NFκB activation and inhibit innate immune activation (29) and thus suppress chronic inflammatory responses. This region contains a putative nuclear localization sequence (amino acids 503-508) as well as a nuclear exit sequence (amino acids 518-526). The absence of these sequences may explain IRAK1c's stability and cytoplasmic localization and possibly its antiinflammatory role. It is therefore tempting to hypothesize that the SLE-associated haplotype block may affect these activities of IRAK1. Clearly, these predictions warrant direct testing in future studies. IRAK1 is located on chromosome Xq28, juxtaposed to a second gene that has also been implicated in SLE susceptibility. A recent study by Sawalha et al. (30) reported the association of the neighboring gene, MECP2, and SLE in Korean and European cohorts. Given the physical proximity of IRAK1 and MECP2 on Xq28, it is plausible that they are in linkage disequilibrium, and the 2 independent studies possibly describe the same genetic association. However, without further reverse genetic studies, it would have been impossible to ascertain whether the disease-causative polymorphism(s) exert their effect through changes in IRAK1 or MECP2. In this regard, the reverse genetic studies presented in this communication shed light on this ambiguity, allowing us to confidently establish that the IRAK1 gene has a critical role in the pathogenesis of SLE. Whether MECP2 is also a causative gene for lupus awaits support from analogous experiments with that gene. Nevertheless, the results we present herein with IRAK1 exemplify the power of combining forward genetic studies in patient cohorts with reverse genetic and functional studies in animal models to elucidate the genetic basis of complex diseases. This powerful bipronged approach can be gainfully used in studies of other genes in SLE and yet other complex genetic diseases.

Although it is too early to suggest the mechanism(s) by which the IRAK1 polymorphisms may alter the disease process in humans, the murine studies presented in this communication suggest an important role for IRAK1 at 2 key checkpoints in lupus development. The first step, which leads to benign serological and cellular autoreactivity, may be the consequence of a breach in central tolerance in the adaptive arm of the immune system, whereas the second step, which leads to pathological autoimmunity, may be mediated by increased activity in the innate arm of the immune system (19, 20, 31). It is remarkable that IRAK1 significantly impacts both checkpoints in lupus development. The likely role of IRAK1 in driving the second checkpoint, myeloid cell hyperactivity, is quite apparent given the central role of IRAK1 in mediating TLR signaling, and hence myeloid cell activation (27). In contrast, the potential role of IRAK1 and TLR signaling at various B cell checkpoints is currently unknown and warrants careful analysis to better understand how IRAK1 might operate in the first checkpoint of lupus development. Conditional deletion of IRAK1 in selected cell types is clearly necessary to address this important gap in our knowledge. Along these lines, future studies elucidating the mechanistic role that IRAK1 might play at both these checkpoints are clearly warranted. The impact of IRAK1 deficiency on other polycongenic models of severe lupus nephritis also needs to be explored.

Several autoimmune disorders are characterized by a strong sex bias, with females being afflicted by SLE almost 10 times more frequently than males. Research efforts over the past 3 decades have implicated sex hormones as being responsible for the sex difference in disease susceptibility. However, effects of sex hormones do not rule out a more direct effect of the X chromosome. Very little is known about whether genes on the sex chromosomes can directly influence SLE susceptibility. Recent reports in mouse models have indicated that genes located on X/Y chromosomes could potentially influence lupus susceptibility (32-34). The present report constitutes the demonstration of a sex chromosome gene in human SLE. The data presented here provide clear evidence that the female predominance of the disease could be attributed, at least in part, to IRAK1 gene dosage by virtue of its location on the X chromosome. The challenge ahead is to fathom the degree to which the sex difference in SLE prevalence can be attributed to X chromosome genes (such as IRAK1) versus hormonal differences.

### Methods

### Recruitment and Biological Sample Collection.

Subjects were enrolled in the Lupus Genetic Study Groups at the University of Southern California and the Oklahoma Medical Research Foundation, in the PROFILE Study Group at the University of Alabama at Birmingham, and from B.L.M., T.J.V., G.S.G., and S.-C.B., using identical protocols. All patients met the revised 1997 American College of Rheumatology criteria for the classification of SLE (35). Ethnicity was self-reported and verified by parental and grandparental ethnicity, when known. Blood samples were collected from each participant, and genomic DNA was isolated and stored by using standard methods. Cases were defined as childhood-onset according to the criterion that the diagnosis of SLE was made before the age of 13 by at least 1 pediatric rheumatologist participating in the study. All protocols were approved by the institutional review boards at the respective institutions.

### Genotyping, Statistical and Stratification Analyses, Immunophenotyping of Mice.

Genotyping. Genotyping was performed using Illumina iSelect Infinium II Assays on the BeadStation 500GX system (Illumina). For analysis, only genotype data from SNPs with a call frequency greater than 90% in the samples tested and an Illumina Gen-Train score greater than 0.7 were used. GenTrain scores measure the reliability of the SNP detection based on the distribution of genotypic classes. The average SNP call rate for all samples was 97.18%. To minimize sample misidentification, data from 91 SNPs that had been previously genotyped on 42.12% of the samples were used to verify sample identity. In addition, at least 1 sample previously genotyped was randomly placed on each Illumina Infinium BeadChip and used to track samples throughout the genotyping process.

Statistical Analyses. Testing for association was completed using the freely available programs SNPGWA (www.phs.wfubmc.edu/web/public┌bios/sec┌gene/downloads.cfm) and PLINK (36). For each SNP, missing data proportions for cases and controls, minor allele frequency, and exact tests for departures from Hardy-Weinberg expectations were calculated. In addition to allelic test of association, the additive genetic model was used as the primary hypothesis of statistical inference. Haploview version 4.0 (37) was used to estimate the linkage disequilibrium between markers and haplotype structures in different ethnicities. Combined p values were calculated from the per-ethnicity p values by using the Fisher method. q values were calculated for different ethnicities by using the q value package (available from http://cran.r-project.org), which implements the q value extension of the false discovery rate (FDR) (38). q values for combined results were calculated by using Benjamini-Hochman formalism (39). q values correspond to the estimation of proportion of false positives among the results. Thus q values ┌0.05 signify ┌5% of false positives and is taken as a measure of significance. Stratification Analyses. To account for potential confounding substructure or admixture in these samples, principal component analyses (PCA) were performed (6) using a large set of SNPs (18,446, which were genotyped on these subjects as part of a larger effort). Four principal components were identified that explained a total of ˜60% of the observed genetic variation. These were used to identify individuals who were genetically distant from other samples in the same ethnic subset, and thus capable of introducing admixture bias. A total of 378 controls and 569 adult SLE and 80 childhood-SLE cases were identified in this fashion and removed from further analysis. After removing these genetic outliers, duplicates, and related samples, 5,457 independent SLE cases and 4,939 controls remained for analysis. We then performed genomic control analysis to calculate the inflation factor using the same set of SNPs. This yielded a λ of 1.13 in European-American samples, 1.03 in Hispanic Americans, 1.08 in African Americans, and 1.04 in Asian Americans. Only the Hispanic sample required a PCA correction to remove the final source of confounding via admixture to obtain the inflation factor given above.

Immunophenotyping of Mice. The different strains were examined at the age of 4-6 months or 9-12 months. The anti-dsDNA, anti-ssDNA, and anti-histone/DNA ELISAs were carried out as described before (16-18). Upon sacrifice, kidneys were fixed, sectioned, and stained with hematoxylin and eosin, and periodic acid Schiff. At least 100 glomeruli were examined per section by light microscopy for evidence of inflammation and/or tissue damage, and graded using the World Health Organization scale, as described before (18), in a blinded fashion. For flow cytometric analysis, splenocytes were depleted of red blood cells by using Tris ammonium chloride, and prepared as single-cell suspensions. The antibodies used to define surface markers on various leukocyte subsets have been described previously (16-18). The mean linear units on the forward scatter channel (FSC) were used as indicators of cell size. DCs were cultured from the BM of different strains and were characterized as detailed elsewhere (20). Statistical comparisons were performed by using the Student t test (SigmaStat, Jandel Scientific).

### Establishing IRAK1-Deficient Lupus Mice.

All mice used were on the C57BL/6 (B6) background. B6.IRAK1^{-/Y}, B6.Sle1^{z}, and B6.Sle3^{z} mice have been characterized previously (15, http://www.ncbi.nlm.nih.gov/pubmed/9502778 http://www.ncbi.nlm.nih.gov/pubmed/110352264-18). B6.IRAK1^{-/Y} mice were bred to B6-based Sle1^{z} or Sle3^{z} lupus congenics to derive F₁ hybrids. The F₁ hybrids were intercrossed to generate F₂ progeny that were then selected for mice that genotyped as B6.Sle3^{z}.IRAK1^{-/Y} or B6.Sle3^{z}.IRAK1^{-/Y}, both strains being homozygous at the respective lupus susceptibility loci. Because IRAK1 is located on the X chromosome, male IRAK1^{-/Y} mice were used as IRAK1-deficient mice, whereas IRAK1^{+/Y} males were used as controls in all experiments. All mice used for this study were bred and housed in a specific pathogen-free colony at the University of Texas Southwestern Medical Center Department of Animal Resources in Dallas, Tex.

### REFERENCES

1. Russ V, et al. In: Dubois' Lupus Erythematosus. Wallace D J, Hahn B H, editors. Philadelphia: Lippincott Williams & Wilkins; 2002. pp. 65-83.
2. Armstrong D L, et al. Function2Gene: A gene selection tool to increase the power of genetic association studies by utilizing public databases and expert knowledge. Bioinformatics. 2008; 9:311-317.
3. Jacob C O, et al. Identification of novel susceptibility genes in childhood-onset systemic lupus erythematosus using a uniquely designed candidate gene pathway platform. Arthritis Rheum. 2007; 56:4164-4173.
4. Cassidy J T. In: Textbook of Pediatric Rheumatology. Cassidy J T, Petty R E, editors. Philadelphia: Elsevier Saunders; 1996. pp. 329-406.
5. Lehman T J A. In: Dubois' Lupus Erythematosus. Wallace D J, Hahn B H, editors. Philadelphia: Lippincott Williams & Wilkins; 2002. pp. 863-884.4.
6. Price A L, et al. Principal components analysis corrects for stratification in genome-wide association studies. Nat Genet. 2006; 38:904-909.
7. Cordell H J, et al. Genetic association studies. Lancet. 2005; 366:1121-1131.
8. Benjamini Y, et al. Controlling the false discovery rate: A practical and powerful approach to multiple testing. J R Stat Soc. 1995; 85:289-300.
9. Graham R R, et al. Three functional variants of IFN regulatory factor 5 (IRF5) define risk and protective haplotypes for human lupus. Proc Natl Acad Sci USA. 2007; 104:6758-6763.
10. Remmers E F, et al. STAT4 and the risk of rheumatoid arthritis and systemic lupus erythematosus. N Engl J. Med. 2007; 357:977-986.
11. Nath S K, et al. A nonsynonymous functional variant in integrin-alpha(M) (encoded by ITGAM) is associated with systemic lupus erythematosus. Nat Genet. 2008; 40:152-154.
12. Oksenberg, J R, et al. The genetics of multiple sclerosis: SNPs to pathways to pathogenesis. Nat Rev Genet. 2008; 9:516-526.
13. Smyth D J, et al. Shared and distinct genetic variants in type 1 diabetes and celiac disease. N Engl J. Med. 2008; 359:2767-2777.
14. Fisher, S A, et al. Genetic determinants of ulcerative colitis include the ECM1 locus and five loci implicated in Crohn's disease. Nat Genet. 2008; 40:710-712.
15. Thomas, J A et al. Impaired cytokine signaling in mice lacking the IL-1 receptor-associated kinase. J Immunol. 1999; 163:978-984.
16. Mohan, C et al. Genetic dissection of SLE pathogenesis: Sle1 on murine chromosome 1 leads to selective loss of tolerance to chromatin components. J Clin Invest. 1998; 101:1362-1372.
17. Mohan C, et al. Genetic dissection of SLE pathogenesis: Sle3 impacts T-cell activation, differentiation and cell death. J Immunol. 1999; 162:6492-6502.
18. Mohan C, et al. Genetic dissection of SLE pathogenesis: A recipe for nephrophilic autoantibodies. J Clin Invest. 1999; 103:1685-1695.
19. Kumar K R, et al. Regulation of B-cell tolerance by the lupus susceptibility gene Ly108. Science. 2006; 312:1665-1669.
20. Zhu J, et al. Genetic dissection of lupus: T-cell hyperactivity as a consequence of hyperstimulatory antigen presenting cells. J Clin Invest. 2005; 115:1869-1878.
21. Martin M U, et al. Summary and comparison of the signaling mechanisms of the Toll/interleukin-1 receptor family. Biochim Biophys Acta. 2002; 1592:265-280.
22. Kollewe C, et al. Sequential autophosphorylation steps in the interleukin-1 receptor-associated kinase-1 regulate its availability as an adapter in interleukin-1 signaling. J Biol Chem. 2004; 279:5227-5236.
23. Baechler E C, et al. The emerging role of interferon in human systemic lupus erythematosus. Curr Opin Immunol. 2004; 16:801-807.
24. Jacob C O, van Der Meide P, McDevitt H O. In vivo treatment of (NZB×NZW)F1 lupus-nephritis with monoclonal antibody to interferon gamma. J Exp Med. 1987; 166:798-802.
25. Wong H K, et al. Abnormal NF-kappaB activity in T lymphocytes from patients with systemic lupus erythematosus is associated with decreased p65-relA protein expression. J Immunol. 1999; 163:1682-1689.
26. Rahman A H, et al. The role of toll-like receptors in systemic lupus erythematosus. Springer Semin Immunopathol. 2006; 28:131-143.
27. Gottipati S, et al. IRAK1: a critical signaling mediator of innate immunity. Cell Signal. 2008; 20:269-276.
28. Li X, et al. IL-1-induced NFkappa B and c-Jun N-terminal kinase (JNK) activation diverge at IL-1 receptor-associated kinase (IRAK). Proc Natl Acad Sci USA. 2001; 98:4461-4465.
29. Rao N, et al. A novel splice variant of interleukin-1 receptor (IL-1R)-associated kinase 1 plays a negative regulatory role in Toll/IL-1R-induced inflammatory signaling. Mol Cell Biol. 2005; 25:6521-6532.
30. Sawalha A H, et al. Common variants within MECP2 confer risk of systemic lupus erythematosus. PLoS ONE. 2008; 3:e1727.
31. Zhu J, Mohan C. SLE 1, 2, 3: Genetic dissection of lupus. in Immune-mediated diseases, from theory to therapy. Adv Exp Med Biol. 2007; 601:85-95.
32. Subramanian S, et al. A Tlr7 translocation accelerates systemic autoimmunity in murine lupus. Proc Natl Acad Sci USA. 2006; 103:9970-9975.
33. Pisitkun P, et al. Autoreactive B cell responses to RNA-related antigens due to TLR7 gene duplication. Science. 2006; 312:1669-1672.
34. Smith-Bouvier D L, et al. A role for sex chromosome complement in the female bias in autoimmune disease. J Exp Med. 2008; 205:1099-1108.
35. Hochberg M C. Updating the American College of Rheumatology revised criteria for the classification of systemic lupus erythematosus. Arthritis Rheum. 1997; 40:1725.
36. Purcell S, et al. PLINK: A toolset for whole-genome association and population-based linkage analysis. Am J Hum Genet. 2007; 81:559-575.
37. Barrett J C, et al. Haploview: analysis and visualization of LD and haplotype maps. Bioinformatics. 2005; 21:263-265.
38. Storey J D, et al. Statistical significance for genomewide studies. Proc Natl Acad Sci USA. 1995; 100:9440-9445.
39. Benjamini Y, et al. Controlling the false discovery rate: A practical and powerful approach to multiple testing. J R Stat Soc B. 1995; 57:289-300.
40. Jacob, C., et al. Identification of IRAK1 as a risk gene with critical role in the pathogenesis of systemic lupus erythematosus. Proc Nati Acad Sci USA. 2009 Apr. 14; 106(15):6256-61. Epub 2009 Mar. 27.

### Example 5

In the past 3 years, genome-wide association (GWA) studies have become extremely popular because they permit the interrogation of the entire human genome, both at levels of resolution earlier unattainable and in thousands of unrelated individuals, while remaining unconstrained by prior hypotheses regarding genetic association with the disease. Although an alternative to GWA studies, pedigree-based linkage analysis, has found disease susceptibility variants, these variants tend to have large relative risks. Furthermore, they have little effect on disease risk at a population level due to their rarity. This argument suggests that more common genetic variants, despite having more moderate relative risk, may be far more important in terms of public health simply because they are more common. GWA studies rely, therefore, on the ‘common disease, common variant’ hypothesis, which suggests that the influences of genetics on many common diseases will be at least partly attributable to a limited number of allelic variants present in more than 1-5% of the population.” But, there also exist examples of rare variants influencing common disease.^{3,4} If multiple rare genetic variants were the primary cause of common complex diseases, GWA studies would have little power to detect them, particularly if allelic heterogeneity existed. Ironically, given the recent huge financial and scientific investment in GWA, there is not a great deal of evidence in support of the common disease, common variant hypothesis.⁵

Furthermore, the GWA approach is also problematic because the massive number of statistical tests performed presents an unprecedented potential for false-positive results, leading to multiple test correction to properly control levels of statistical significance, coupled with the increased need for replication of findings.⁶ If performed appropriately, correction for multiple testing will render most of the findings insignificant because of the large number of tests (≧300 000, typically).

Given that the case-control samples for GWA usually number in thousands, it might be expected that such studies are well powered. However, several authors have shown that, given the strict genome-wide significance criteria that studies must fulfill, the power of such studies is much less than might be naively imagined.^{7,8}

There is also a limit to how large population-based studies can get due to constraints such as budget, time and the physical number of cases in the population; so there may be a further class of variants that are too rare to be captured by GWA, but are not sufficiently at high risk to be captured by population-based linkage (for example, Cambien and Tiret⁹). Alternative approaches are needed to find these variants.

To counteract these shortcomings of GWA, we have adopted a Bayesian approach that concentrates on a collection of candidate pathways rather than concentrating on specific candidate genes (or the whole genome). Using these pathways, we have taken advantage of the accumulated data from pre-existing association studies of adult systemic lupus erythematosus (SLE) families, candidate gene investigations, information gained from genetics of mouse models of lupus and the gene expression profiling data of human SLE to identify sets of genes and regions containing genes that have a higher prior likelihood of association with SLE.

To implement this approach, we have developed a set of programs that embody a combination of automated and manual approaches to maximize the power of gene-association studies using prior information to select and prioritize genes, both to decrease the size of the problem and to increase the likelihood of discovering reproducible association.¹⁰

Utilizing this bioinformatic-driven design, we selected ˜10 000 SNPs derived from 1000 genes on a custom-made platform to genotype a modest sample of 753 subjects corresponding to 251 childhood-onset SLE trios (SLE patient and both parents).” Family-based transmission disequilibrium test and multi-test correction analysis identified SELP and IRAK1 as novel SLE-associated genes with high degree of significance corrected for multiple tests using the false discovery rate (FDR) less than 0.05. Importantly, the original study had also identified a number of genes that although not significant by the accepted criteria were considered to be noteworthy for further investigation (0.05<FDR<0.8). We present here the results on a group of 10 such genes (109 SNPs), obtained in case-control study with a large number of subjects.

### Results

In this study, we explored the SLE association of 10 promising genes, each of which showed an FDR of <0.8 in our earlier transmission disequilibrium test-based study. The candidate genes evaluated in this study are BCL6, CASP10, interleukin (IL)-16, IRF5, KIR2DS4, KLRGI, PRL, PTPN22, protein tyrosine phosphatase receptor type T (PTPRT) and toll-like receptor (TLR)8. This case-control study included an independent childhood-onset cohort of 769 childhood-onset SLE and 5337 cases of adult-onset SLE subjects and 5317 healthy controls, each being composed of four ethnicities (64).

An important component of our approach was the deliberate recruitment and usage of childhood-onset SLE cases. They present a unique subgroup of patients for genetic study because the earlier disease onset, a more severe disease course, a greater frequency of family history of SLE and a lesser effect of sex hormones in disease development.^{12,13} imply a higher genetic load or a more penetrant expression of this genetic load. However, because childhood-onset SLE may also show the involvement of different genetic factors relative to adult onset disease, we analyzed childhood-onset and adult-onset groups of SLE patients separately.

To account for any potential confounding substructure or admixture, we performed principal component analyses¹⁴ detailed in materials and methods. Excluding the outliers identified by principal component analyses resulted in low inflation factors in all ethnicities except Hispanic Americans, with only the latter requiring additional PC correction.

As we are performing tests of multiple related hypotheses, controlling for study-wide significance is an important concern to avoid promulgating false positives due to the multiple testing. A classical correction for multiple testing is the Bonferroni correction (or similar family-wise error-rate corrections). Unfortunately, it is both too strict and inappropriate in studies such as the present one because of the underlying assumption that each test is independent, whereas in actuality a complex and unknown interdependence is present among SNPs in linkage disequilibrium (LD).^{11,15} In light of this, we have instead calculated an estimate of the FDR that measures the number of false positives (type I errors) we would have to accept to consider a result a true discovery (reject the null hypothesis), using the Benjamini and Hochberg¹⁶ procedure, considering the total number of SNPs tested and the four different ethnic groups. Combined P-values were calculated from the per-ethnicity P-value using the Fisher's method. 28 SNPs from 7 genes out of the 10 tested have significant combined association with SLE in adult- or childhood-onset subgroups after correction for multiple testing. Importantly, these genes include not only the earlier associated genes, PTPN22 and IRF5, but also several novel genes that have not yet been associated with SLE. We did not find significant association with the SNPs genotyped in KIR2DS4, PRL and BCL6 in either childhood- or adult-onset SLE. With the exception of rs2476601 in PTPN22, none of the SNPs that we found to be significant code for amino-acid changes; only rsl1073001 in IL-16 is in an exon, but this variant does not encode for a different amino acid. The most significant SNP found was rs4728142 in IRF5, with a combined P-value in adults on the order of 10⁻²⁹ and a corresponding FDR on the order of 10⁻²⁷.

SNPs from four novel genes, KLRG1, IL-16, PTPRT and TLR8 are associated, with SLE in four ethnic groups (European Americans (EA), African Americans (AA), Asian Americans (AsA) and Hispanic Americans (HA)) in childhood- and adult-onset SLE cases (64). It is noteworthy that the majority of the significantly associated SNPs show significance in multi-pie ethnicities both in adult-onset and in childhood-onset SLE. Nevertheless, it is also important to notice cases in which SLE association is strongly ethnicity dependent. For example, the SNPs around exon 1 of TLR8 are not significant in AsA but are significant in HA, both in children and in adults. These graphs also show the distribution of significant: SNPs in the genes. For example, the significant SNPs in IL-16 are concentrated around exon 18.

Next we performed haplotype analyses in different ethnic groups, children and adults separately (64). Haplotype blocks in KLRG1, are noticeably different in the various ethnicities (64). Interestingly, no significant haplotype blocks were found in adult EA. The significant haplotype blocks in IL-16 are limited to childhood-onset HA (64). The significant haplotype blocks in PTPRT were limited to AsA and a smaller block in childhood-onset HA.

IRF5 has a large number of significant haplotype blocks that are similar in the various ethnicities besides AA (64). Comparing our results with the earlier published data on IRF5 association with SLE, we found that rs729302 SNP was reported to be associated with SLE in an EA population with a P-value of 4×10^{-4 17} or 5.2×10^{-7 18} in Swedish cohort with a P-value of 2.7×10⁻⁴ (not corrected for multitest)¹⁹ and in family trios (uncorrected P-value of 5.0×10⁻⁴)²⁰. We confirmed these findings on an EA cohort with a multitest-corrected FDR of 3.4×10⁻⁹ in adults and 1.8×10⁻⁸ in childhood-onset cases (64). Furthermore, we found a significant association of this rs729302 SNP with SLE in HA adults (Q-value of 8.0×10⁻³) and combined children (FDR of 1.6×10⁻⁵), but not in AA or AsA cohorts in either adult- or childhood-onset SLE (64). The previously reported association of rs4728142 in a Swedish cohort¹⁹ and family trios²⁰ was confirmed by us and extended to all four ethnicities in adult-onset, and to all ethnicities, except AA in childhood-onset disease (64). We have also confirmed the involvement of rs2004640 in EA,¹⁷⁻¹⁹ African Americans,¹⁸ Chinese²¹ and family trios,²⁰ and in both childhood- and adult-onset SLE in each ethnicity, except for childhood-onset HA. Association with rs752637 in Europeans was shown by some earlier investigators^{18,19} but not by others.¹⁷ Our studies found a strong association of this SNP with SLE in EA adults (FDR 1.4×10⁻¹⁰), but not as strong in adult HA, AsA or children EA cohorts.

We have confirmed the earlier association of rs3807306 with SLE showed in a European cohort¹⁹ and in EA and AA¹⁸ and extended this association to HA and AsA. The association of rs3807306 in AA was not significant in our study (Q˜value of 0.09), but with an uncorrected P-value of 0.03, it does not contradict the results of an earlier study.¹⁸ Also, in agreement with Sigurdsson et al.,¹⁹ we did not detect association of rs1874328 with SLE (64). Underscoring the ethnic dependence of many SNP associations, rs3807135, found earlier to be SLE associated in a family trio study,²⁰ was found by us to be associated in adults only in EA and HA, but not in AsA or AA, with a very low Q-value of 0.51 for adult-onset AA (64). We have also confirmed SLE-associated haplotype block in the same region as reported earlier^{17,18} and extended this block in chromosome region and detected its SLE association with other ethnicities (64).

Significant haplotype blocks in TLR8, are distributed throughout the gene, though childhood-onset EA has a haplotype with much higher significance located in the 5′-UTR of TLR8. In addition, the LD between SNPs that compose the haplotype blocks of TLR8 in adult-onset AA and childhood-onset AA and in EA. The differences in LD structure, lead to distinct haplotype blocks observed in different ethnicities.

Although no SNPs found in BCL6 survived the multitest correction, the haplotype analysis indicated that a haplotype block in childhood-onset AsA is significantly associated with the disease (64), underlining the utility of haplotype analysis even in the absence of singly significant SNPs.

### Discussion

Using a large cohort of adult- or childhood-onset SLE cases in four different ethnicities and comparable numbers of relevant controls, we show in this study that seven genes exhibit a significant (FDR<0.05) association with SLE, both confirming some genes that were earlier found to be associated with SLE (PTPN22 and IRF5) and novel findings (KLRGI, IL-16, PTPRT, TLR8 and CASP10) that were not reported earlier. Furthermore, although none of the SNPs within the BCL6 gene achieved significant association with SLE after multitest correction, a haplotype block within BCL6 shows significant association with the disease as well. These genes are additional to IRAK1 and SELP, which we found to be significantly associated with SLE in our first step study,¹¹ and their follow-up studies are being reported separately.

The presented results show the powerful potential of using a two-step Bayesian approach utilizing up-to-date biotechnology and bioinformatic methods for discovering novel genes. This methodology yielded more novel significant results than the much more expensive GWA approach. Indeed, Iles⁵ re-examined the results of 54 studies across 22 different relatively common complex diseases, most of which were GWA studies with some GWA follow-up studies. Only 45 disease-associated SNPs found initially in GWA studies could be conclusively ascertained as significant. Furthermore, for several diseases, such as Parkinson's disease,²² bipolar disorder^{23,24} and hypertension,²³ no new replicable variant has yet been found using GWA (as of February 2008). Compared with approximately two new genes identified per disease using GWA, our Bayesian approach yielded many more novel genes. As a case in point, several of the genes discovered using the Bayesian design were missed by the GWA studies performed to date in SLE.²⁵⁻²⁸

GWA studies have been praised because of their unbiased nature, namely they are ‘unbiased by prior assumptions about the DNA alterations responsible’.²⁹ However, it does not make much sense to ignore the whole universe of valuable information collected about the pathogenesis of a common disease that has been studied for decades by excellent investigators. The earlier studies, whereas often not resulting (or not even designed) to identify SLE-associated genes, did provide a well-documented background to reveal SLE-associated physiological pathways. Accordingly, our design took advantage of the vast literature on SLE in humans and in mouse models of lupus. The results obtained show that using prior available information as a primary guide allows one to identify novel SLE-associated genes with high confidence.

In each of these novel genes, there is much biological information to form hypotheses as to their involvement in the genetic predisposition to SLE. Thus, the association of KLRG1 (killer cell lectin-like receptor 1) gene (mapped at 12p12) implicates the involvement of NK cells in the genetic predisposition to SLE. KLRG1 is expressed on NK cells and on subsets of activated T cells. KLRG1-expressing NK cells show decreased proliferative activity³⁰ SLE patients, including childhood-onset cases, have quantitative and qualitative alterations in NK cells.³¹⁻³³ The association of SLE with KLRG1 showed in our studies, coupled to earlier findings, that first-degree relatives of SLE patients³³ and healthy monozygotic co-twins of SLE patients³⁴ display reduced numbers and activity of NK cells, suggesting that this latter phenotype might be involved in disease causation rather than being simply a consequence of the disease process.

More recent work has shown that KLRG1 expression defines a novel and distinctive subset of CD4+ Treg cells that depend on IL-2 and express FoxP3 but are only partially overlapping with the CD4+ and CD25+ Treg subset.³⁵ Interestingly, the cytokine IL-16, shown to be elevated in SLE subjects,^{36,37} is a natural ligand of the CD4+ molecule and induces CD4 T-cell anergy.^{38,39} IL-16 may also induce or recruit CD4+ FoxP3+ T regs in the tissue.⁴⁰° Thus, the involvement of IL-16 in the genetic predisposition to SLE as shown here might be in the same pathway as KLRG1.

SLE is characterized by the production of autoantibodies to certain cellular macromolecules, such as the small nuclear ribonucleoprotein particles (snRNPs),⁴¹ and by the increased expression of type I interferon (IFNA).^{42,43} Conserved RNA sequences within snRNPs can stimulate TLR7 and −8, as well as activate innate immune cells, such as plasmacytoid dendritic cells, which respond by secreting high levels of IFNA. Possibly, SLE patients' sera containing autoantibodies to snRNPs form immune complexes that are taken up through the Fc receptor gammaRII and efficiently stimulate plasmacytoid dendritic cells to secrete IFNAs. Thus, a prototype autoantigen, the snRNP, can directly stimulate innate immunity, suggesting that autoantibodies against snRNP may initiate the autoimmune response by stimulating TLR7/8.⁴¹ IFNA, by inducing genes such as IRF5, can exert major effects on the immune system, including inducing a TH1 response and maintaining T-cell activation, while also lowering the threshold for B-cell activation and promoting B-cell survival and differentiation.⁴⁴ It is likely that genetic variants that change IRF5 activity could result in a prolongs pro-inflammatory response and/or potentially break immunological tolerance. It is, therefore, possible that the genetic involvement of TLR8 gene may at least partially overlap with the IFNA-induced gene, IRF5, in predisposing to SLE.

Importantly, IRF5 signaling has also been shown to play a role in the regulation of cell cycle and apoptosis,⁴⁵ raising the possibility that susceptibility variants of IRF5 may affect SLE pathogenesis at the level of the apoptosis pathway as well.⁴⁴ Indeed, the involvement of defective apoptosis in the predisposition of SLE is well documented.^{46,47} The association of CASP10 with SLE shown in this paper may further emphasize the importance of apoptosis pathways in the genetic predisposition of SLE. The CASP10 genes locus at 2q23 is mutated in human autolymphoproliferative syndrome type II.⁴⁸ Patients with autolymphoproliferative syndrome II exhibit prominent non-malignant lymphadenopathy, hepatosplenomegaly, hyperimmunegammaglobulinemia with multiple autoantibodies, autoimmune hemolytic anemia and lymphocytosis with accumulation of normally rare CD4−/CD8− T cells⁴⁸ as in the lupus-prone MRL//lpr/lpr mice. Importantly, CASP10 is not only involved in Fas signaling but is also essential for apoptosis signaling through multiple death receptors.⁴⁹

Although further studies will be necessary to prove the involvement of BCL6 in the pathogenesis of SLE, a significant haplotype block within this gene is an important first step in incriminating this transcriptional repressor. BCL6, a frequently translocated oncogene in diffuse large B-cell lymphoma, has also an important function in regulating the differentiation of B cells, T cells and myeloid cells.⁵⁰ More specifically, BCL6 is required for germinal center formation and is also a critical inhibitor of Th2 responses and infianlmation.^{51,52}

Protein tyrosine phosphatase receptor type T together with the previously associated PTPN22^{25,53-55} underscore the importance of PTPs in the pathogenesis of SLE. PTPRT has been characterized as a key inhibitor of STAT-3,⁵⁶ which, in turn, mediates transcriptional activation in response to several cytokines involved in the inflammatory response, such as IL-6.

Interestingly, PTPRT is a genetic locus that was suggested to be associated with rheumatoid arthritis in three independent GWA studies.^{23,57,58} In each of these GWA studies, the SNPs within PTPRT lost their significance after multitest correction, further exemplifying the problematic of GWA studies.

In summary, the extensive involvement of these candidate genes in the regulation of the immune response makes their association with SLE potentially very important and justifies subsequent genetic and functional studies.

### Materials and Methods

Recruitment and biological sample collection. Subjects were enrolled in the Lupus Genetic Study Groups at USC and OMRF, in the PROFILE Study Group at UAB and from additional collaborators using identical protocols. All patients met the revised 1997 ACR criteria for the classification of SLE.⁵⁹ Ethnicity was self-reported and verified by parental and grandparental ethnicity, when known. Blood samples were collected from each participant, and genomic DNA was isolated and stored using standard methods. Cases were defined as childhood onset according to the criterion that the diagnosis of SLE was made before the age of 13 years by at least one pediatric rheumatologist participating in the study. All protocols were approved by the Institutional Review Boards at the respective institutions.

Genotyping. Genotyping was performed using Illumina iSelect Infinium II Assays on the BeadStation 500GX system (Illumina, San Diego, Calif., USA). For analysis, only genotype data from SNPs with a call frequency greater than 90% in the samples tested and an Illumina GenTrain score greater than 0.7 were used. GenTrain scores measure the reliability of the SNP detection based on the distribution of genotypic classes. The average SNP call rate for all samples was 97.18%. To minimize sample misidentification, data from 91 SNPs that had been genotyped earlier on 42.12% of the samples were used to verify sample identity. In addition, at least one sample genotyped earlier was randomly placed on each Illumina Infinium BeadChip and used to track samples throughout the genotyping process.

Statistical analyses. Testing for association was completed using the freely available R module, snpassoc⁶⁰ and PLINK.⁶¹ For each SNP, missing data proportions for cases and controls, minor allele frequency and exact tests for departures from Hardy-Weinberg expectations were calculated. In addition to allelic test of association, the additive genetic model was used as the primary hypothesis of statistical inference. Haploview version 4.0⁶² and the R module genetics (available from http://cran.r-project.org/web/packages/genetics/index.html) were used to estimate the LD between markers and haplotype structures in different ethnicities.

Combined P-values were calculated from the per-ethnicity P-values using Fisher's method. FDR estimates using Q-values were calculated for different ethnicities using the Q-value package (available from http://cran.r-project.org), which implements the Q-value extension of FDR.⁶³ The FDR for combined results were estimated using Benjamini and Hochberg¹⁶ procedure, as the proportion of correctly, rejected null hypotheses was possibly overestimated when using the Q-value extension, and this, procedure provides a more conservative estimation of FDR (but with less power). The FDR corresponds g the proportion of false positives among the results. Thus, an estimate of FDR less than 0.05 signifies that less than 5% of the results accepted as true are false positives and is taken as a measure of significance.

Stratification analyses. To account for potential confounding substructure or admixture in these samples, principal component analyses were performed¹⁴ using a large set of SNPs (18446, which were genotyped on these subjects as part of a larger effort). Four principal components were identified that explained a total of ˜60% of the observed genetic variation. These were used to identify individuals who were genetically distant from other samples in the same ethnic subset, and thus capable of introducing admixture bias. A total of 378 controls and 569 adult SLE and 80 childhood-SLE cases were identified in this fashion and removed from further analysis a (64). After removing these genetic outliers, duplicates and related samples, 5457 independent SLE cases and 4939 controls remained for analysis. We then performed genomic control analysis to calculate the inflation factor λ using the same set of SNPs. This yielded a λ of 1.13 in European American samples, 1.03 in Hispanic Americans, 1.08 in African Americans and 1.04 in Asian Americans. Only the Hispanic sub-population required a principal component analysis correction to remove the final source of confounding through admixture to obtain the inflation factor given above.

### REFERENCES

1. Collins F S, et al. Variations on a theme: cataloging human DNA sequence variation. Science 1997; 278: 1580-1581.
2. Pearson T A, et al. How to interpret a genome-wide association study. JAMA: 2008; 299: 1335-1344.
3. Cohen J C, et al. Multiple rare alleles contribute to low plasma levels of HDL cholesterol. Science 2004; 305: 869-872.
4. Romeo S, et al. Population-based resequencing of ANGPTL4 uncovers variations that reduce triglycerides and increase MDL. Nat Genet 2007; 39: 513-516.
5. lies M M. What can genome-wide association studies tell us about the genetics of common disease? PLoS Genet 2008; 4: e33.
6. Hunter D J, et al. Drinking from the fire hose-statistical issues in genomewide association studies. N Engl J Med 20.07; 357: 436-439.
7. Wang W Y, B et al. Genome-wide association studies: theoretical and practical concerns. Nat Rev Genet 2005; 6: 109-118.
8. Jorgenson B, et al. Coverage and power in genomewide association studies. Am J Hum Genet 2006; 78: 884-888.
9. Cambien F, et al. Genetics of cardiovascular diseases: from single mutations to the whole genome. Circulation 2007; 116: 1714-1724.
10. Armstrong D L, et al. Function2Gene: a gene selection tool to increase the power of genetic association studies by utilizing public databases and expert knowledge. BMC Bioinformatics 2008; 9: 311-317.
11. Jacob C O, et al. Identification of novel susceptibility genes in childhood-onset Systemic Lupus Erythematosus using a uniquely designed candidate gene pathway platform. Arthritis Rheum 2007; 56: 4164-4173.
12. Cassidy J T In: Cassidy J T, Petty R E (eds). Textbook of Pediatric Rheumatology. Elsevier Saunders: Philadelphia, 1996, pp 329-406.
13. Lehman T J A In: Wallace D J, Hahn B H (eds). Dubois' Lupus Erythematosus. Lippincott Williams & Wilkins: Philadelphia, 2002, pp 863-884.
14. Price A L, Pa et al. Principal components analysis corrects for stratification in genome-wide association studies. Nat Genet 2006; 38: 904-909.
15. Cordell H J, et al. Genetic association studies. Lancet 2005; 366: 1121-1131.
16. Benjamini Y, et al. Controlling the false discovery rate: a practical and powerful approach to multiple testing. J Stat Soc 1995; 85: 289-300.
17. Grahnm R R, et al. A common haplotype of interferon regulatory factor 5 (IRF5) regulates splicing and expression and is associated with increased risk of systemic lupus erythematosus Nat Genet 2006; 38: 550-555.
18. Kelly J A, et al. Interferon regulatory factor-5 is genetically associated with systemic lupus erythematosus in African Americans. Genes Immun 2008; 9: 187-194.
19. Sigurdsson S, et al. Comprehensive evaluation of the genetic variants of interferon regulatory factor 5 (IRF5) reveals a novel 5 bp by length polymorphism as strong risk factor for systemic lupus erythematosus. Hum Mol Genet 2008; 17: 872-881.
20. Graham R R, et al. Three functional variants of IFN regulatory factor 5 (IRF5) define risk and protective haplotypes for human lupus. Proc Natl Acad Sci USA 2007; 104: 6758-6763.
21. Siu H O, et al. Association of a haplotype of IRF5 gene with systemic lupus erythematosus in Chinese. J Rheumatol 2008; 35: 360-362.
22. Maraganore D M, et al. High-resolution whole-genome association study of Parkinson disease. Am J Hum Genet 2005; 77: 685-693.
23. Wellcome Trust Case Control Consortium. Genome-wide association study of 14,000 cases of seven common diseases and 3,000 shared controls. Nature 2007; 447: 661-678.
24. Baum A E, et al. A genome-wide association study implicates diacylglycerol kinase eta (DGKH) and several other genes in the etiology of bipolar disorder. Mol Psychiatry 2008; 13: 197-207.
25. Harley J B, et al. Genome-wide association scan in women with systemic lupus erythematosus identifies susceptibility variants in ITGAM, PXK, KIAA1542 and other loci. Nat Genet 2008; 40: 204-210.
26. Horn G, et al. Association of systemic lupus erythematosus with C8orf13-BLK and ITGAM-ITGAX. N Engl J Med 2008; 358: 900-909.
27. Kozyrev S V, et al. Functional variants in the B-cell gene BANK1 are associated with systemic lupus erythematosus. Nat Genet 2008; 40: 211-216.
28. Graham R R, et al. Genetic variants near TNFAIP3 on 6q23 are associated with systemic lupus erythematosus. Nat Genet 2008; 40: 1059-1061.
29. Altshuler D, et al. Guilt beyond a reasonable doubt. Nat Genet 2007; 39: 813-814.
30. Voehringer D, et al. Lack of proliferative capacity of human effector and memory T cells expressing killer cell lectinllke receptor G1 (KLRG1). Blood 2002; 100: 3698-3702.
31. Erkeller-Yusel F, et al. Lymphocyte subsets in a large cohort of patients with systemic lupus erythematosus. Lupus 1999; 2: 227-231.
32. Yabuhara A, et al. A killing defect of natural killer cells as an underlying immunologic abnormality in childhood systemic lupus erythematosus. J Rheumatol 1996; 23: 171-177.
33. Green M R, K et al. Natural killer cell activity in families of patients with systemic lupus erythematosus: demonstration of a killing defect in patients. Clin Exp Immunol 2005; 141: 165-173.
34. Stohl W, et al. Impaired recovery and cytolytic function of CD56+ T and non-T cells in systemic lupus erythematosus following in vitro polyclonal T cell stimulation. Studies in unselected patients and monozygotic disease-discordant twins. Arthritis Rheum 1996; 39: 1840-1851.
35. Beyersdorf N, et al. Characterization of mouse CD4T cell subsets defined by expression of KLRG1. Eur J Immunol 2007; 37: 3445-3454.
36. Lee S, et al. Circulating interleukin-16 in systemic lupus erythematosus. Br J Rheumatol 1998; 37: 1334-1337.
37. Lard L R, et al. Elevated IL-16 levels in patients with systemic lupus erythematosus are associated with disease severity but not with genetic susceptibility to lupus. Lupus 2002; 11: 181-185.
38. Theodore A C, et al. CD4 ligand IL-16 inhibits the mixed lymphocyte reaction. J Immunol 1996; 157: 1958-1964.
39. Klimiuk P A, et al. IL-16 as an antiinflammatory cytokine in rheumatoid synovitis. J Immunol 1999; 162: 4293-4299.
40. McFadden C, et al. Preferential migration of T regulatory cells induced by IL-16. J Immunol 2007; 179: 6439-6445.
41. Vollmer J, et al. Immune stimulation mediated by autoantigen binding sites within small nuclear RNAs involves Toll-like receptors 7 and 8. J Exp Med 2005; 202: 1575-1585.
42. Baechler E C, et al. Interferon-inducible gene expression signature in peripheral blood cells of patients with severe lupus. Proc Natl Acad Sci USA 2003; 100: 2610-2615.
43. Bennett L, et al. Interferon and granulopoiesis signatures in systemic lupus erythematosus blood. J Exp Med 2003; 197: 711-723.
44. Rhodes B et al. The genetics of SLE: an update in the light of genome-wide association studies. Rheumatol 2008; 47: 1603-1611.
45. Barnes B J, K et al. Interferon regulatory factor 5, a novel mediator of cell cycle arrest and cell death. Cancer Res 2003; 63: 6424-6431.
46. Mevorach D. Systemic lupus erythematosus and apoptosis: a question of balance. Clin Rev Allergy Immunol 2003; 25: 49-60.
47. Mehrian R, et al. Synergistic effect between IL-10 and bcl-2 genotypes in determining susceptibility to systemic lupus erythematosus. Arthritis Rheum 1998; 41: 596-602.
48. Wang J, et al. Inherited human Caspase 10 mutations underlie defective lymphocyte and dendritic cell apoptosis in autoimmune lymphoproliferative syndrome type II. Cell 1999; 98: 47-58.
49. Müllauer L, et al. Mutations in apoptosis genes: a pathogenetic factor for human disease. Mutat Res 2001; 488: 211-231.
50. Staudt L M, et al. Regulation of lymphocyte cell fate decisions and lymphomagenesis by BCL6. Int Rev Immunol 1999; 18: 381-403.
51. Dent A L, S et al. Control of inflammation, cytokine expression, and germinal center formation by BCL6. Science 1997; 276: 589-592.
52. Ye B H, et al. et al. The BCL6 proto-oncogene controls germinal-centre formation and Th2-type inflammation. Nat Genet 1997; 16: 161-170.
53. Kyogoku C, et al. Genetic association of the R620W polymorphism of protein tyrosine phosphatase PTPN22 with human SLE. Am J Hum Genet 2004; 75: 504-507.
54. Wu H, et al. Association analysis of the R620W polymorphism of protein tyrosine phosphatase PTFN22 in systemic lupus erythematosus families: increased T allele frequency in systemic lupus erythematosus patients with autoimmune thyroid disease. Arthritis Rheum 2005; 52: 2396-2402.
55. Lee Y H, et al., The PTPN22 C1858T functional polymorphism and autoimmune diseases-a meta-analysis. Rheumatology 2007; 46: 49-56.
56. Zhang X, et al. Identification of STAT3 as a substrate of receptor protein tyrosine phosphatase T. Proc Natl Acad Sci USA 2007; 104: 4060-4064.
57. Julià A, et al. Genome-wide association study of rheumatoid arthritis in the Spanish population: KLF12 as a risk locus for rheumatoid arthritis susceptibility. Arthritis Rheum 2008; 58: 2275-2286.
58. Plenge R M, et al. Two independent alleles at 6q23 associated with risk of rheumatoid arthritis. Nat Genet 2007; 39: 1477-1482.
59. Hochberg M C. Updating the American College of Rheumatology revised criteria for the classification of systemic lupus erythematosus. Arthritis Rheum 1997; 40: 1725.
60. Gonzàlez J R, et al. SNPassoc: an R package to perform whole genome association studies. Bioinformatics 2007; 23: 644-645.
61. Purcell S, et al. PLINK: a toolset for whole-genome association and population-based linkage analysis. Am J Hum Genet 2007; 81: 559-575.
62. Barrett J C, et al. Haploview: analysis and visualization of LD and haplotype maps. Bioinformatics 2005; 21: 263-265.
63. Storey J D, Tibshirani R. Statistical significance for genomewide studies. Proc Natl Acad Sci USA 1995; 100: 9440-9445.
64. Armstrong, D., et al. Genes and Immunity 2009 July; 10(5):446-56. Epub 2009 May 14.

What is claimed is:

1. A method of diagnosing an autoimmune disease in a child comprising detecting the presence of 2 or more genes that are associated with child-onset autoimmune disease.

2. The method according to claim 1, wherein the genes are associated with child-onset autoimmune disease.

3. The method according to claim 1, wherein the autoimmune disease is Systemic Lupus Erythmeatosus (SLE).

4. The method according to claim 1, wherein the genes are SELP, IRAK1, KLRG1, TNFSF4, TNFRSF6, TLR8, or Fas.

5. A method of diagnosing an autoimmune disease in an adult comprising detecting the presence of 2 or more genes that are associated with adult-onset autoimmune disease.

6. The method according to claim 5, wherein the genes are associated with adult-onset autoimmune disease.

7. The method according to claim 5, wherein the autoimmune disease is SLE.

8. The method according to claim 5, wherein the genes are IRAK1, KLRG1, TNFSF4, or FAIM.

9. A method of diagnosing an autoimmune disease in a subject comprising detecting the presence of 2 or more genes that are associated with autoimmune disease.

10. The method according to claim 9, wherein the genes are associated with both child- and adult-onset autoimmune disease.

11. The method according to claim 9, wherein the autoimmune disease is SLE.

12. The method according to claim 9, wherein the genes are SELP, IRAK1, KLRG1, IRF5, STAT4, TNFRSF6, TNFSF4, PTPN22, TLR8, FAIM, IRF5, or NCF2.
